(19) United States
(12) Patent Application Publication
(10) Pub. No.: US 2017/0000887 A1
(43) Pub. Date: Jan. 5, 2017

(54) UNIFORM CORE-SHELL TIO2 COATED UPCONVERSION NANOPARTICLES AND USE THEREOF

(71) Applicant: NATIONAL UNIVERSITY OF SINGAPORE, Singapore (SG)

(72) Inventors: Yong Zhang, Singapore (SG); Zhengquan Li, Singapore (SG); Niagara Muhammad Idris, Singapore (SG); Khee Chee Soo, Singapore (SG); Sasidharan Swarnalatha Lucky, Singapore (SG)

(21) Appl. No.: 15/105,834

(22) PCT Filed: Dec. 30, 2014
(86) PCT No.: PCT/SG2014/000623

§ 371 Date: Jun. 17, 2016

Related U.S. Application Data
(60) Provisional application No. 61/923,858, filed on Jan. 6, 2014.

(51) Int. Cl. A61K 41/00; A61N 5/06; A61K 47/48; A61K 9/51; A61K 33/24

(52) U.S. Cl. CPC A61K 41/008; A61K 9/5115; A61K 33/24; A61K 47/48884; A61K 47/48215; A61K 47/48061; A61K 47/48861; A61N 5/062; A61N 2005/0662; A61N 2005/0659

(57) ABSTRACT

An upconversion nanoparticle (UCN) coated with a layer of semiconductor material is disclosed. The UCN core acts as a nanotransducer to convert near infrared (NIR) to visible and/or ultraviolet (UV) light while the semiconductor shell serves as a photocatalyst. Upon excitation by NIR light, the UCN upconverts NIR light to UV and/or visible light of different wavelengths. Spectral overlap between the emitted UV and absorption wavelength of the coated TiO₂ activates the TiO₂ layer to generate cytotoxic reactive oxygen species (ROS), which can be used in photodynamic therapy for the treatment of cancer cells. Stability and uptake of the nanoparticles can be increased by altering the coating of the nanoparticle, such as by a polymer and a dispersion stabilizer.

### RELATED APPLICATIONS

This application claims the benefit of U.S. Provisional Application No. 61/923,858, filed on Jan. 6, 2014. The entire teachings of the above application are incorporated herein by reference.

### BACKGROUND OF THE INVENTION

Traditional fluorophores are mainly based on ‘downconversion fluorescence’: these emit low energy fluorescence when excited by a high energy light, typically within the ultraviolet (UV)-short wavelength visible range. The opposite effect to downconversion also exists, a process called upconversion. The upconversion luminescence arises from a phenomenon whereby light of low energy, usually in the near-infrared (NIR) range, is converted to light of higher energy in the shorter wavelength range in an anti-Stokes emission process. Upconversion fluorescent materials are usually made of host lattices of nanocrystals such as LaF₃, YF₃, Y₂O₃, LaPO₄, or NaYF₄ doped with trivalent lanthanide ions such as Yb³⁺, Er³⁺ and Tm³⁺. An important feature is their characteristically narrow photoluminescence spectra¹ whose emission color can be fine-tuned by doping different concentrations of the lanthanide ions² to give varying colors in the UV, visible and NIR range upon excitation by 980 nm NIR light. In addition, an upconversion process is much more efficient than two-photon absorption and is thus conveniently excited using inexpensive and commercially available continuous wave (CW) laser diodes³. Another unique feature of these upconversion fluorescent materials is their exceptional photostability coupled with low photodamage (NIR ray being non-harmful) to cells and delicate proteins that allows long-term live imaging and photoactivation to be possible^{4,5}. Equally attractive is the near-zero background fluorescence associated with the use of these materials since most other materials, including biological molecules, do not possess this upconverting property⁶. When such background-free property is combined with their high quantum yield³, upconversion fluorescent materials may be a highly promising class of luminescent probes that can offer astounding detection sensitivity for tracing minute amount of target molecules. Besides, the NIR light used to excite these materials can pass deep into tissues⁷, thereby affording non-invasive imaging and photoactivation at deep tissue level. More importantly, the inert core elements and relatively less toxic incorporated rare earth lanthanides⁸ that formulate upconversion fluorescent materials provides a particular advantage over highly toxic metals such as cadmium-selenium used in quantum dots, thus providing a safe avenue for use in in vivo and clinical settings.

Current photodynamic therapy (PDT) drugs are activated by UV or visible light in the spectral regions below 700 nm which has a limited penetration depth in tissue. Conventional PDT is thus limited to flat, superficial tumors or tumors that are accessible to endoscopes, whereas solid organ tumors are at a thickness much greater than the depth of penetration possible with the current technology. In order to overcome the depth limitation of conventional PDT using UV/visible light and be able to ablate a bulky solid tumor or deep-seated tumor, the use of light in the NIR region (700-1100 nm) can be used to achieve maximum tissue penetration depth. Light of NIR wavelengths is absorbed less by epidermal melanin, undergoes less light scattering than light of lower wavelengths, and penetrates deeper into human skin dermis and blood than visible light. Light within this spectral region has been shown to penetrate tissue at depths beyond one centimeter with no observable damage to the intervening tissue. Thus PDT using NIR light to activate the photosensitizers, a process referred to herein as NIR-PDT, can be extended to the treatment of thick, bulky or deep-seated tumors, providing broader treatment options for various tumor sizes, types and locations. As almost all the photosensitizers currently marketed for PDT are activated by UV/visible light only, using NIR light in PDT requires a light transducer to convert deeply penetrating low-energy NIR light to higher-energy, shorter UV/visible wavelengths to match the activation absorption spectrum of these photosensitizers at depth. There remains a need to develop a near-IR photodynamic therapy drug that will overcome the limitations of conventional photodynamic therapy by offering a higher penetration depth of NIR light, and that will further upconvert said NIR light to UV/Visible light. In particular, NIR-PDT drugs capable of generating multiple types of reactive oxygen species are particularly important in the development of new photodynamic therapies, because such drugs enable a multi-pronged mechanism of destroying target cells.

### SUMMARY OF THE INVENTION

The present invention relates to an upconversion nanoparticle (UCN) coated with a layer of titanium dioxide (TiO₂). The UCN core acts as a nanotransducer to convert NIR to visible and ultraviolet (UV) light while the TiO₂ shell serves as a photocatalyst. Upon excitation by just a single wavelength of NIR light, the UCN can upconvert NIR to UV and visible light of different wavelengths. Spectral overlap between the emitted UV and absorption wavelength of the coated TiO₂ activates the TiO₂ layer to generate cytotoxic reactive oxygen species (ROS).

### BRIEF DESCRIPTION OF THE DRAWINGS

The foregoing will be apparent from the following more particular description of example embodiments of the invention, as illustrated in the accompanying drawings.

FIGS. 1A-D show TiO₂-coated NaYF₄:Yb,Tm UCN. FIG. 1A is a transmission electron microscopy image of the TiO₂—UCN (scale bar, 50 nm). FIG. 1B is a fluorescence emission spectrum of the TiO₂—UCN under 980 nm NIR laser excitation. Inset shows the absorption spectrum of pure TiO₂, (arbitrary units (a.u.)). FIG. 1C shows ROS production from TiO₂—UCN under 980 nm NIR irradiation (2.16 W/cm²) as determined by fluorescence of a ROS fluorogenic marker—aminophenyl fluorescein (APF). APF fluorescence is plotted as a function of exposure time (t) to the 980 nm NIR laser. Values are means±s.e.m. (the experiments for each group were run in duplicate). FIG. 1D is a schematic illustration on the mechanism of TiO₂—UCN to generate ROS (not to scale). After excitation by a NIR light at 980 nm, the UCN upconvert the NIR light to UV and visible light of different wavelengths, as seen in FIG. 1B. Spectral overlap between the emitted UV light and the maximum absorption wavelength of the coated TiO₂ activates the TiO₂ shell to generate an electron-hole (e⁻-h⁺) pair due to its electron being excited from the valence band to the conduction band. The resultant hole oxidizes a water (H₂O) molecule to yield hydroxyl radical (.OH), while the electron reduces oxygen (O₂) to give superoxide anion (.O₂⁻) or hydrogen peroxide (H₂O₂).

FIGS. 2A-2C show stability of TiO₂—UCN fluorescence. Fluorescence emission spectrum of NIR-irradiated TiO₂—UCN suspension in (FIG. 2A) different physiological solutions, (FIG. 2B) water for different time duration and (FIG. 2C) acidic water for different time duration.

FIGS. 3A-3E show the type of ROS produced from NIR-irradiated TiO₂—UCN suspension in water. Presence of (FIG. 3A) hydrogen peroxide, (FIG. 3B) hydroxyl radical, (FIG. 3C) superoxide anion and (FIG. 3D) singlet oxygen species produced was detected by fluorescence quenching of the APF dye upon addition of their scavengers, sodium pyruvate, dimethyl sulfoxide (DMSO), Tiron and sodium azide, respectively, into the suspension of TiO₂—UCN irradiated with the 980 nm NIR laser (2.16 W/cm²). APF fluorescence is plotted as a function of exposure time (t) to the 980 nm NIR laser. Values are means±s.e.m. (the experiments for each group were run in duplicate). *P<0.05 compared to NIR-irradiated TiO₂—UCN without scavenger addition. FIG. 3E shows a verification study on singlet oxygen production from NIR-irradiated TiO₂—UCN by fluorescence of a fluorogenic marker specific for singlet oxygen-singlet oxygen sensor green dye. The dye fluorescence is plotted as a function of exposure time (t) to the 980 nm NIR laser.

FIG. 4 demonstrates the shelf life of TiO₂—UCN stored as dry powder at room temperature (RT). TiO₂—UCN activity for ROS production under 980 nm NIR irradiation (2.16 W/cm²) for 60 min was monitored at different days of storage at RT for over 2 months by APF fluorescence and plotted as a function of storage days.

FIG. 5 shows ROS production from TiO₂—UCN stored as dry powder under different conditions for 20 days. TiO₂—UCN activity for ROS production under 980 nm NIR irradiation (2.16 W/cm²) before storage and after 20 days of storage at either 4° C. in the dark, RT in the dark or RT but exposed to light was determined by APF fluorescence and plotted as a function of exposure time (t) to the 980 nm NIR laser.

FIG. 6 demonstrates the shelf life of TiO₂—UCN stored as dry powder at 4° C. TiO₂—UCN activity for ROS production under 980 nm NIR irradiation (2.16 W/cm²) for 60 min was monitored at different days of storage at 4° C. for over 2 months by APF fluorescence and plotted as a function of storage days. Values are means±s.e.m. (the experiments for each group were run in duplicate).

FIG. 7 shows ROS production from TiO₂—UCN stored as dry powder at 4° C. versus −20° C. for 4 days (P>0.05 between 4° C. and −20° C.). TiO₂—UCN activity for ROS production under 980 nm NIR irradiation (2.16 W/cm²) was determined by APF fluorescence and plotted as a function of exposure time (t) to the 980 nm NIR laser.

FIG. 8 demonstrates the stability of TiO₂—UCN activity for ROS production when soaked in water. ROS production from 980 nm NIR-irradiated TiO₂—UCN (at 2.16 W/cm² for 60 min) soaked in water for up to 24 h was monitored by APF fluorescence and plotted as a function of time soaked in the water. Values are means±s.e.m. (the experiments for each group were run in duplicate). *P<0.05 compared to 0 h soaked in water.

FIGS. 9A-9C demonstrate the stability of TiO₂—UCN activity for ROS production when soaked in water after storage under different conditions for 24 h. ROS production from 980 nm NIR-irradiated TiO₂—UCN (at 2.16 W/cm² for 60 min) (FIG. 9A) before soaking and (FIG. 9B) after 24 h soaked in water at either 4° C. in the dark, RT in the dark or RT but exposed to light was determined by APF fluorescence and plotted as a function of exposure time (t) to the 980 nm NIR laser. (FIG. 9C) Drop in TiO₂—UCN activity for ROS production is charted as the percentage of APF fluorescence after soaking for 24 hours in water (shown in FIG. 9B) divided by the APF fluorescence before soaking (shown in a) for each of the different storage condition.

FIG. 10 shows the stability of TiO₂—UCN activity for ROS production after being soaked overnight in different solvents. ROS production from 980 nm NIR-irradiated TiO₂—UCN (at 2.16 W/cm²) before soaking and after soaked overnight in either ethanol (EtOH), tetrahydrofuran (THF), toluene, chloroform or anhydrous DMSO was determined by APF fluorescence and plotted as a function of exposure time (t) to the 980 nm NIR laser. The respective solvents were removed and the TiO₂—UCN was resuspended in water just prior to the ROS test. Values are means±s.e.m. (the experiments for each group were run in triplicate). *P<0.05 compared to before soaking.

FIGS. 11 A-11E illustrate (FIG. 11A) a schematic of synthesis of TiO₂—UCN and Mal-PEG-TiO₂—UCN; Transmission electron microscope (TEM) images of (FIG. 11B) TiO₂—UCNs and (FIG. 11C) Mal-PEG-TiO₂—UCN, (scale bar: 50 nm); (FIG. 11D) Fluorescence emission spectra of 100 μg/ml TiO₂—UCN in phosphate buffered saline (PBS) under NIR excitation at different power densities (photon multiplier tube (PMT) voltage at 500V); inset shows fluorescence emission from TiO₂—UCNs upon NIR excitation at 90 W/cm²; (FIG. 11E) Fourier transform infrared (FT-IR) absorption spectra of TiO₂—UCN, Maleimide-PEG-silane and Maleimide-PEG-silane conjugated TiO₂—UCN (Mal-PEG-TiO₂—UCN).

FIGS. 12A-12B illustrate (FIG. 12A) TEM of UCN (NaYF₄:Yb,Tm) core, silica coated UCN (NaYF₄:Yb,Tm@SiO₂) and TiO₂—UCN before annealing (TiO₂ coated NaYF₄:Yb,Tm@SiO₂), scale bar: 20 nm; and (FIG. 12B) Fluorescence emission spectra of 300 μg/ml TiO₂—UCN in PBS under NIR excitation at power densities equivalent to that used for in vitro (2.1 W/cm²) and in vivo (0.5 W/cm²) PDT (PMT voltage at 950V).

FIGS. 13A-13F illustrate the characteristics of TiO₂—UCNs and Mal-PEG-TiO₂—UCN. FIG. 13A shows the average hydrodynamic size of 100 μg/ml TiO₂—UCN soaked in different media at RT plotted as a function of time the nanoparticles were soaked, *P<0.0001 versus size of TiO₂—UCNs in RPMI without fetal bovine serum (FBS). FIG. 13B shows the effect of addition of 10% FBS on hydrodynamic size of 100 μg/ml TiO₂—UCN soaked in PBS, *P<0.0001 compared to size of TiO₂—UCNs at 24 h. FIG. 13C shows the average hydrodynamic size of 100 μg/ml Mal-PEG-TiO₂—UCN in different solutions at RT plotted as a function of time. FIG. 13D shows the image of a silver stained gel to detect adsorption of serum proteins on the surface of nanoparticles suspended in either RPMI with 10% FBS or in 100% FBS. Unmodified TiO₂—UCN in water and 100% FBS were run as negative and positive controls, respectively. FIG. 13E is a comparison of ROS production from non-irradiated and irradiated nanoparticles in PBS, *^{#}P<0.0001 versus ROS production from non-irradiated TiO₂—UCNs and Mal-PEG-TiO₂—UCNs respectively after 60 min. Data are mean (n>2)±SD. FIG. 13F is a comparison of ROS production from 1 mg/ml Mal-PEG-TiO₂—UCNs irradiated with NIR or UV light in the presence of tissue phantoms of different thickness, *P<0.05 versus ROS.

FIGS. 14A-14F illustrate characteristics of TiO₂—UCNs and Mal-PEG-TiO₂—UCN such as (FIG. 14A) Average poly dispersity index (PDI) of 100 μg/ml TiO₂—UCN soaked in different media at RT plotted as a function of time the nanoparticles were soaked. FIG. 14B shows the effect of addition of 10% FBS on PDI of 100 μg/ml TiO₂—UCN soaked in PBS. FIG. 14C shows the average hydrodynamic size of TiO₂—UCN soaked in different physiological solutions at 37° C. monitored up to 24 h by dynamic light scattering and plotted as a function of time. Data are mean (n>2)±SD. FIG. 14D shows the difference in aggregate formation of 100 μg/ml TiO₂—UCNs soaked in PBS at 6 h, 24 h as well as PBS with 10% serum at 24 h (FIG. 14E) Average PDI of 100 μg/ml Mal-PEG-TiO₂—UCN in different solutions at RT plotted as a function of time and (FIG. 14F) Photograph of stable dispersion of 100 μg/ml Mal-PEG-TiO₂—UCNs soaked in PBS at 0, 6 and 24 h.

FIGS. 15A-15F illustrate the in vitro uptake of TiO₂—UCNs of the present invention. FIG. 15A shows representative fluorescence microscope images of upconversion fluorescence (column 3) indicating nanoparticle uptake by macrophages following 1 h incubation with 1 mM of respective nanoparticles; columns 1 and 2 are fluorescence indicating cell membrane and nucleus respectively (scale bar: 50 μm). FIG. 15B shows the Comparison of fluorescence intensities of nanoparticles at a concentration of 1 mM taken up by macrophages following 1 h incubation; *P=0.0004. Data are mean fluorescence intensities (n>3)±SD. FIG. 15C shows the comparison of titanium content by ICP-AES analysis following uptake of 1 mM TiO₂—UCN or Mal-PEG-TiO₂—UCN in oral squamous cell carcinoma (OSCC) cells; *P<0.0001. Data are mean concentration of titanium (n=2)±SD. FIG. 15D shows a comparison of fluorescence intensities of 1 mM TiO₂—UCNs surface modified with methoxy-PEG-silane (Met-PEG-TiO₂—UCN) or maleimide-PEG-silane (Mal-PEG-TiO₂—UCN) incubated with OSCC cells; *P<0.05. Data are mean fluorescence intensities (n>3)±SD. FIG. 15E shows the relative UCN fluorescence intensity showing influence of N-ethylmaleimide (NEM) on the cellular uptake of Mal-PEG-TiO₂—UCNs, OSCC cells were preincubated with or without 1 nM NEM in serum-free RPMI for 15 mins, followed by 6 h incubation with 1 mM Mal-PEG-TiO₂—UCNs (Data are mean (n<6)±SD, *P=0.0464). FIG. 15F shows the Influence of serum proteins in the binding and internalization of 1 mM Mal-PEG-TiO₂—UCNs in OSCC cells following 3 h incubation; *P=0.0091. Data are mean fluorescence intensities (n>3)±SD.

FIGS. 16A-16D illustrate (FIG. 16A) in vitro dark toxicity of nanoparticles incubated with OSCC cells for 6 h using MTT, *P=0.0082. FIG. 16B shows in vitro dark toxicity of nanoparticles incubated with normal human fibroblast (NHF) cells using MTT assay; data are mean (n=3)±SD. FIG. 16C in vitro dark toxicity of nanoparticles incubated with OSCC cells for 6 h using trypan blue assay; data are mean (n=3)±SD. FIGS. 16C and 16D show the rate of hemolysis in red blood cells (RBCs) upon 2 h incubation with nanoparticles at incremental concentrations, *P<0.05, **P<0.0001; data are expressed as mean±SD and n=4.

FIG. 17 is a bar graph of optimization of NIR laser dose for in vitro PDT. OSCC cells treated with 1 mM Mal-PEG-TiO₂—UCN were irradiated with varying power and fluence of the 980 nm NIR laser. Light alone control was also run alongside by irradiating the cells that were not treated with any nanoparticles, with the same power and light fluence. Response of the cells to the PDT treatment was then quantified by measuring their viability. NIR laser power at 1.2 W delivering a light fluence of 675 J/cm² giving the lowest cell viability with negligible cell death caused by NIR light alone (*P<0.0001), was chosen as the optimum NIR laser dose for all subsequent in vitro PDT studies. Data are mean (n>3)±SD.

FIGS. 18 A-18C illustrate comparisons of TiO₂—UCNs vs Mal-PEG-TiO₂—UCNs. FIG. 18A shows OSCC cell viability 24 h following in vitro PDT in the presence of nanoparticles; Control cells are untreated cell assumed to have 100% viability, Light alone control are cells treated with NIR light alone. Data are mean cell viability % (n=3)±SD. FIG. 18B shows the extent of ROS generated was quantified based on the fluorescence intensity of carboxy-DCF (Data are mean relative fluorescence intensities (n=3)±SD). FIG. 18C shows bright-field live cell images showing mechanism of cell death in OSCC cells counterstained with trypan blue, inset shows cells undergoing necrosis (scale bar: 10 μm).

FIGS. 19A-19C illustrate variation in the cell uptake, cell viability and in vitro ROS generation ability of 1 mM Mal-PEG-TiO₂—UCN incubated with OSCC cell for either 1 h or 6 h. FIG. 19A shows fluorescence microscopy images showing blue upconversion fluorescence indicating the presence of Mal-PEG-TiO₂—UCNs. Mal-PEG-TiO₂—UCNs were predominantly localized in the cell membrane (arrows) when they are incubated for a shorter period (1 h) with OSCC cells (Scale bar: 20 μm). FIG. 19B shows a comparison of cell viability post NIR-PDT at 675 J/cm² following incubation of 1 mM Mal-PEG-TiO₂—UCNs at 1 h and 6 h; *P<0.5 (Data are mean (n=3)+SD). FIG. 19C shows a comparison of in vitro ROS generation following PDT in the presence of 1 mM Mal-PEG-TiO₂—UCNs that were incubated for 1 h and 6 h. Images showing green fluorescence indicating positive staining for the fluorogenic marker for ROS, carboxy-DCF (Scale bar: 25 μm).

### DETAILED DESCRIPTION OF THE INVENTION

A description of example embodiments of the invention follows.

A “nanocomposite” refers to a nanoparticle conjugated to a second material that modifies, for example, the function, activity, or structure of a nanoparticle. For example, the second material enables the nanoparticle to be used in certain applications in which the nanoparticle would not be used if not for the presence of the second material. In certain embodiments, a second material is coated on the nanoparticle. In further embodiments, the size of the nanocomposite is from about 30 to about 200 nm. In particular embodiments of the invention, the nanocomposite is a nanoparticle coated by an intermediate coating layer, which is in turn coated by a layer of semiconductor material. A linking molecule, for example 3-aminopropyl trimethoxysilane, is optionally deposited on the intermediate coating layer, underneath the semiconductor layer.

The term “upconversion” as used herein refers to a material that converts low-energy light of high wavelength (e.g. near infrared light) to high energy light of low wavelength (e.g., visible or UV light).

“Near infrared light” refers to light having a wavelength between 700 nm and 1100 nm. “Visible light” refers to light having a wavelength between 400 nm and 700 nm. “Ultraviolet light” refers to light having a wavelength between 100 nm and 400 nm.

A “semiconductor” as used herein, is a material having electrical conductivity that falls between a metal and an insulator. A semiconductor has an electronic band structure consisting of a lower energy “valence band” which is partially filled by electrons separated by a band gap from a higher energy “conduction band”.

A “reactive oxygen species” as used herein, includes hydrogen peroxide, hydroxyl radical, superoxide anion, singlet oxygen, nitric oxide, peroxyl radical and peroxynitrile anion.

A “linking group” as used herein, is an atom or a small molecule used to link two or more groups. In an example embodiment, a linking group connects a targeting agent through a series of covalent bonds to an upconversion nanoparticle (UCN) coated with a layer of semiconductor material. The upconversion nanoparticle (UCN) coated with a layer of semiconductor material is alternately referred to herein as a nanocomposite. The linking group can be attached to the surface of the semiconductor coating layer. In certain embodiments, the linking group is polyethylene glycol.

In an alternate example embodiment, the linking group is used to link a UCN with a coating layer. An example of one such linking group is (3-aminopropyl)-trimethoxysilane (APS). In alternate embodiments, the linking group is attached to or alternately deposited on an intermediate coating layer that is applied to the surface of the nanoparticle underneath the semiconductor shell layer. In certain embodiments, the intermediate coating layer is a silica-based composition, which can include SiO₂ and a partially hydrolyzed silica such as H₂SiO₃, H₄SiO₄, H₂Si₂O₅, H₆Si₂O₇ and SiH₄O₄.

A linking group, for example polyethylene glycol, can confer some advantages to the semiconductor-coated UCN, such as improving dispersion of the nanocomposite in an aqueous environment as well as enhancing the circulatory lifetime of the nanocomposite material so that it has a greater chance of reaching the targeted tumor sites. In certain embodiments, the linking molecule, for example APS, confers a positive charge on the surface of the upconversion nanoparticle or on the surface of the coating, for example an intermediate SiO₂ coating. Conferring a charge on the surface of a particle has the advantage of preventing it from agglomerating with other particles due to the repulsive interaction between similarly charged particles. Accordingly, such charged particles achieve monodispersibility in solutions. Monodispersibility enables the semiconductor material to coat individual nanoparticles rather than a collection of agglomerated nanoparticles, which would result in the formation of an agglomerated mass of semiconductor-coated UCN. In further embodiments, the positive charge on the surface of the nanoparticles directs the hydrolysis of TiO₂ precursors on the surface coating layer of SiO₂-coated UCN, such that a homogeneous and uniform layer of the TiO₂ shell is coated on the surface of each nanoparticle. In some embodiments, the hydrolysis step partially hydrolyzes the silica intermediate layer, forming a layer comprising H₂SiO₃ in addition to SiO₂. Accordingly, in an example embodiment, the linking group can be a dispersion stabilizer. The term “dispersion stabilizer” refers to a moiety comprising a hydrophobic end and a hydrophilic end which, when associated to a nanoparticle, deters the nanoparticles from aggregating in solution. An example dispersion stabilizer is polyethylene glycol (PEG).

A “targeting agent”, as used herein, means a structure that has an affinity for a biological target in in vitro or in vivo applications. A biological target includes cell surface receptors. In further embodiments, a biological target is an antigen, a protein, or a peptide.

An “antibody” as used herein, is a protein that identifies and neutralizes a foreign body, for example, a bacterium or a virus. The antibody can be polyclonal or monoclonal, and the term “antibody” is intended to encompass both polyclonal and monoclonal antibodies. The terms polyclonal and monoclonal refer to the degree of homogeneity of antibody preparation, and are not intended to be limited to particular methods of production. The term “antibody” as used herein also encompasses functional fragments of antibodies, including fragments of chimeric, humanized, primatized, veneered or single chain antibodies. Functional fragments include antigen-binding fragments. Such fragments can be produced by enzymatic cleavage or by recombinant techniques. For example, papain, pepsin or other protease with the requisite substrate specificity can also be used to generate fragments. Antibodies can also be produced in a variety of truncated forms using antibody genes in which one or more stop codons has been introduced upstream of the natural stop site.

An “affibody”, as used herein, is a small protein that acts as an antibody mimic. In certain embodiments, affibodies have binding affinity for particular large proteins or peptides.

The term “aptamer” encompasses both oligonucleic acid aptamers and peptidic aptamers. In certain embodiments, aptamers have binding affinity for small molecules, proteins, nucleic acids, cells, tissues and organisms. In certain other embodiments, aptamers interfere with biological processes, for example protein interactions within a cell.

The term “treat” or “treatment” or “treating” refers to inhibiting the progress of, or preventing the disorder or condition being addressed. Treatment, as used herein, can also refer to reduction of a number of targets. In an example embodiment, a target or biological target is a cancer cell, and treatment reduces the number of cancer cells.

The notation “NP@C”, for example, NaYF₄@TiO₂, is used in the application to denote a nanoparticle, for example NaYF₄, having a semi-conductor coating, for example a coating made of TiO₂.

The invention is related to the use of uniform upconversion nanoparticle (UCN) coated with an continuous layer of titanium dioxide (TiO₂) as a light transducer to convert deeply penetrating and safe low-energy near infrared (NIR) light to higher-energy, shorter visible and ultraviolet (UV) wavelengths that matches the activation absorption spectrum of the coated TiO₂ to generate reactive oxygen species (ROS) for use in diverse applications such as photodynamic therapy (PDT), treatment of waste water, sterilizing and deodorizing surfaces in homes and hospitals, development of hydrophilic surfaces as self-cleaning and anti-fogging coatings and cleavage of peptides and proteins. The upconversion nanoparticle coated with semiconductor material is useful in synergistic non-invasive imaging and photodynamic treatment of large and deep-seated tumors. Using NIR light in photodynamic therapy helps overcome the limitations of conventional PDT by offering a higher penetration depth of NIR light and upconversion of nanoparticles to convert NIR to UV/visible light.

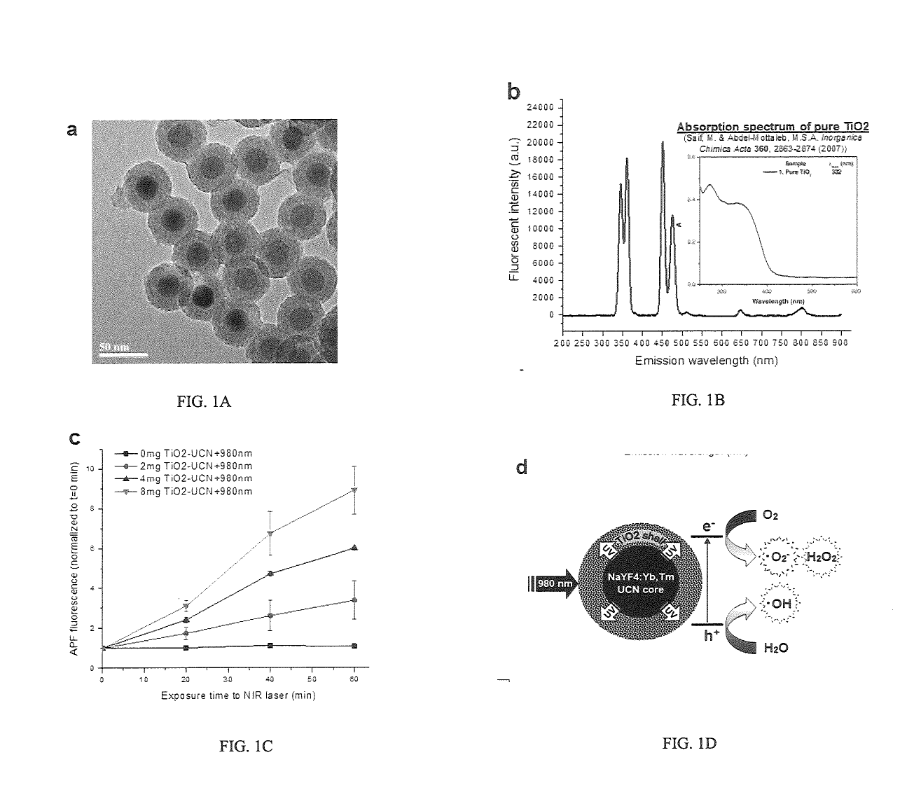

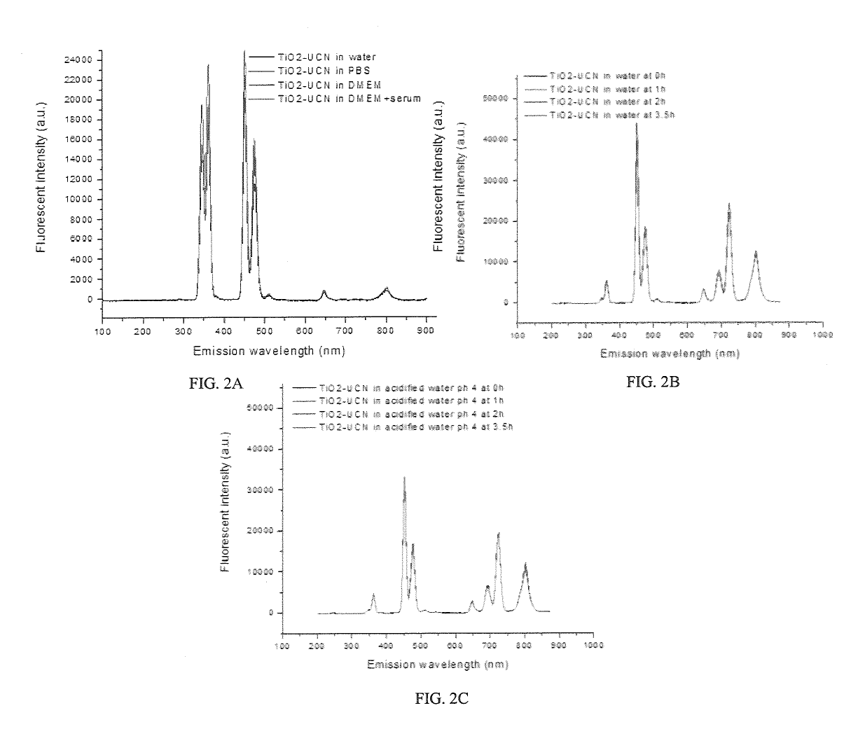

Upconversion nanoparticles such as NaYF₄ nanocrystals co-doped with lanthanide ions Yb and Tm forms the core of the nanoconstruct while a layer of TiO₂ is coated on the core surface to yield the resultant core-shell structured TiO₂—UCN (FIG. 1A). Here, the UCN core acts as a nanotransducer to convert deeply penetrating NIR to visible and UV light while the TiO₂ shell serves as a photocatalyst. Upon excitation by just a single 980 nm wavelength light, the UCN can up-convert NIR to UV and visible light of different wavelengths (FIG. 1B). Spectral overlap between the emitted UV and absorption wavelength of the coated TiO₂⁹ (FIG. 1B inset) activate the TiO₂ layer to generate several cytotoxic reactive oxygen species (ROS) (FIG. 1C) including hydroxyl radical, superoxide anion and hydrogen peroxide that can be used to kill cancer cells for PDT or kill microorganisms for sterilizing and deodorizing surfaces in homes and hospitals, break down organic pollutants in the remediation of waste water as well as cleave peptides and proteins (FIG. 1D). Investigation on the stability of these TiO₂—UCN to fluoresce under 980 nm NIR excitation when suspended in different physiological solutions such as water, PBS and DMEM culture medium with or without serum supplementation, revealed no obvious difference in the intensity of their emission fluorescence (FIG. 2A). Interestingly, this photostability feature of the particles were also observed when they are soaked in acidic water with a pH of 4 for up to 3.5 h (FIG. 2C), albeit showing a small drop in fluorescence intensity when compared to soaking in water (with a pH of ˜6.5) for the same time duration (FIG. 2B). This may find useful implications when the particles are endocytosed into an acidic compartment of the cell such as endosomes and lysosomes in subsequent downstream biological applications.

As shown previously in FIG. 1C, TiO₂—UCN successfully produced ROS upon NIR irradiation. Here, the dye aminophenyl fluorescein (APF) was used as a ROS indicator. The dye APF remained nonfluorescent until they become oxidized upon reacting with a ROS molecule, resulting in the emission of a bright green fluorescence. This fluorescence emission of the dye APF in water was measured in the presence of different amount of TiO₂—UCN irradiated at 980 nm for 20 min intervals and saw an obvious increase in the APF dye fluorescence with a corresponding increase in the amount of irradiated TiO₂—UCN (FIG. 1C). To determine the type of ROS that is produced from these, NIR-irradiated TiO₂—UCN suspended in water, a series of scavenger experiments was performed.

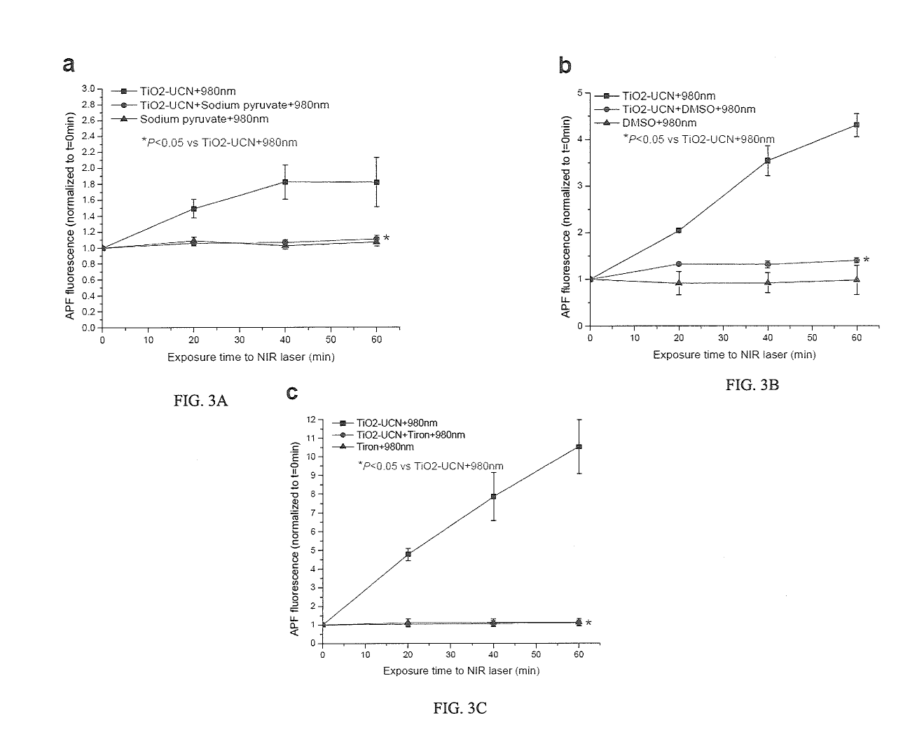

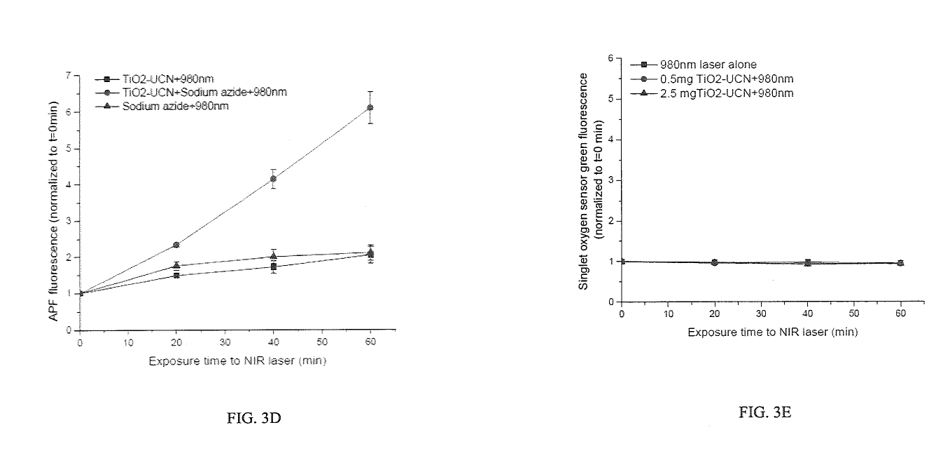

The respective scavengers for hydrogen peroxide, hydroxyl radical, superoxide anion and singlet oxygen, sodium pyruvate, DMSO, Tiron and sodium azide, were individually added into the suspension of TiO₂—UCN irradiated with the 980 nm NIR laser. Fluorescence quenching of the APF dye upon addition of these respective scavengers would denote presence of the particular type of ROS. As shown in FIGS. 3A-3C, significant fluorescence quenching was seen in samples containing the scavengers sodium pyruvate, DMSO and Tiron (P=0.02328, 0.00766 and 0.00317, respectively, compared to irradiated TiO₂—UCN without scavenger addition), while that having the scavenger sodium azide showed no such quenching of the APF dye (FIG. 3D). This indicates that photoinduced TiO₂—UCN produced more than one type of ROS and that includes hydrogen peroxide, hydroxyl radical and superoxide anion. Although the result suggests that singlet oxygen was not generated, fluorescence enhancement of the APF dye observed upon addition of the scavenger sodium azide is rather surprising. To circumvent possible confounding results, the study was verified by another test that used a fluorogenic marker specific only towards singlet oxygen—the singlet oxygen sensor green dye. The test detected no fluorescence increase of the singlet oxygen sensor green dye from NIR-irradiated TiO₂—UCN suspension (FIG. 3E), thus corroborating previous result from the scavenger experiment in that no singlet oxygen was produced from the NIR-irradiated TiO₂—UCN. Indeed, this is in contrast to conventional PDT that kills cancer cells by singlet oxygen produced upon activation of photosensitizer by direct visible light. This may have implication on the mechanism in which TiO₂—UCN-mediated PDT kills cells and that it may be different from conventional PDT.

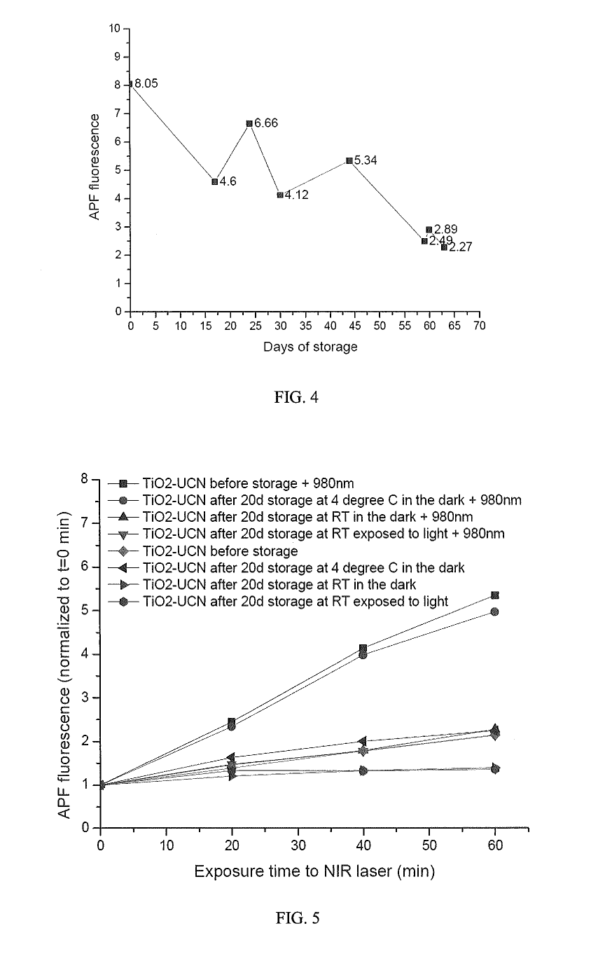

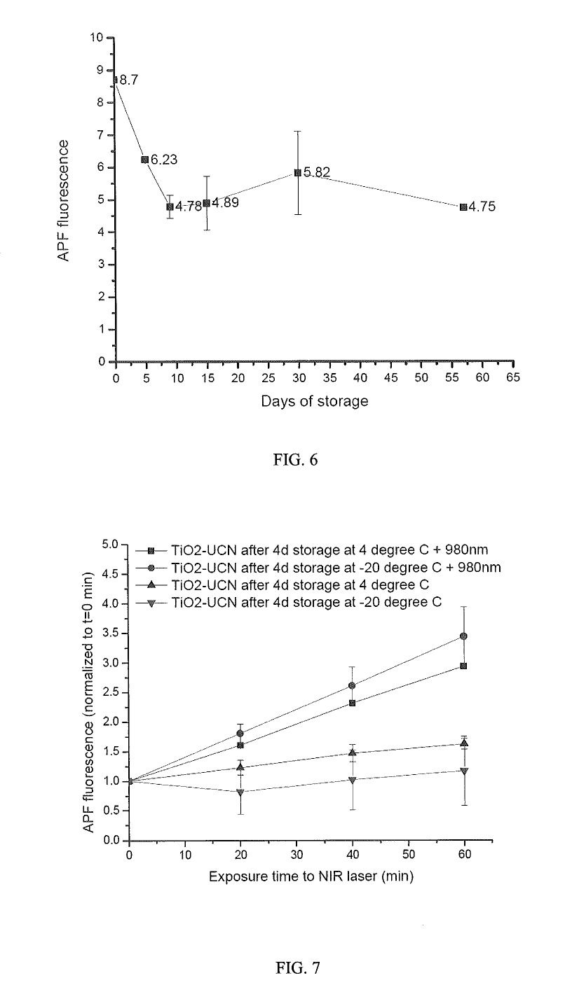

As storage conditions may have an effect on TiO₂—UCN activity for ROS production, this section is devoted to investigating the shelf life of the TiO₂—UCN. TiO₂—UCN activity for ROS production under 980 nm NIR irradiation was monitored as it aged over 2 months at room temperature (RT) when stored as dry powder. A drop in the TiO₂—UCN activity for ROS production was clearly observed over time under this storage condition (FIG. 4). Following this observation, an attempt to search for an optimal storage condition that best preserved the TiO₂—UCN activity for ROS production was made. A short experiment was designed in which the nanoparticles were stored for only 20 days as dry powder under different storage conditions of either 4° C. in the dark, RT in the dark or RT but exposed to light (FIG. 5). Aging of the TiO₂—UCN powder at 4° C. in the dark for 20 days did not correlate to a marked decrease in its activity for ROS production. Indeed, this is in stark contrast to storing it at RT either in the dark or with light exposure for the same time duration that seemed to drastically lower its activity for ROS production. With the optimum condition to best preserve the TiO₂—UCN activity for ROS production being now identified, the shelf life of the nanoparticles stored under this condition was next monitored for about 2 months. Although there was a fair bit of decline in the TiO₂—UCN activity for ROS production in the first 10 days of storage, its level of activity became stabilized thereafter for up to 57 days of storage. It is worth to note here that the nanoparticles' level of activity for ROS production was not significantly different at 57 days of storage compared to that at 0 day of storage (P=0.10247 between 0 day and 57 days of storage) (FIG. 6). Further attempt to investigate on whether lower temperature would influence the TiO₂—UCN activity for ROS production was also made by storing the nanoparticles as dry powder at −20° C. for 4 days. When compared to those stored at 4° C. for the same time duration, no appreciable difference was observed in the TiO₂—UCN activity for ROS production (P=0.4211 between storage at 4° C. and −20° C.) (FIG. 7). It seems then that storage of the TiO₂—UCN powder in the fridge is sufficient in preserving its shelf life and that lower temperature such as that in the freezer is not necessarily better in this sense.

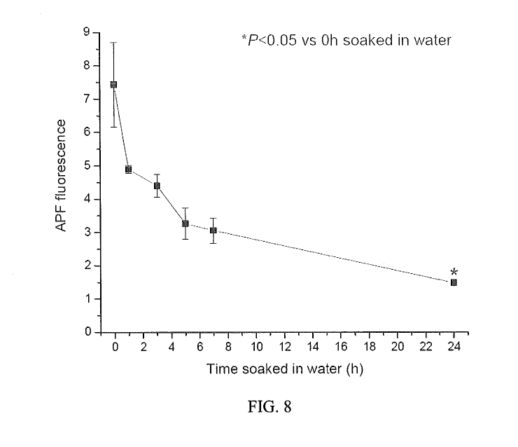

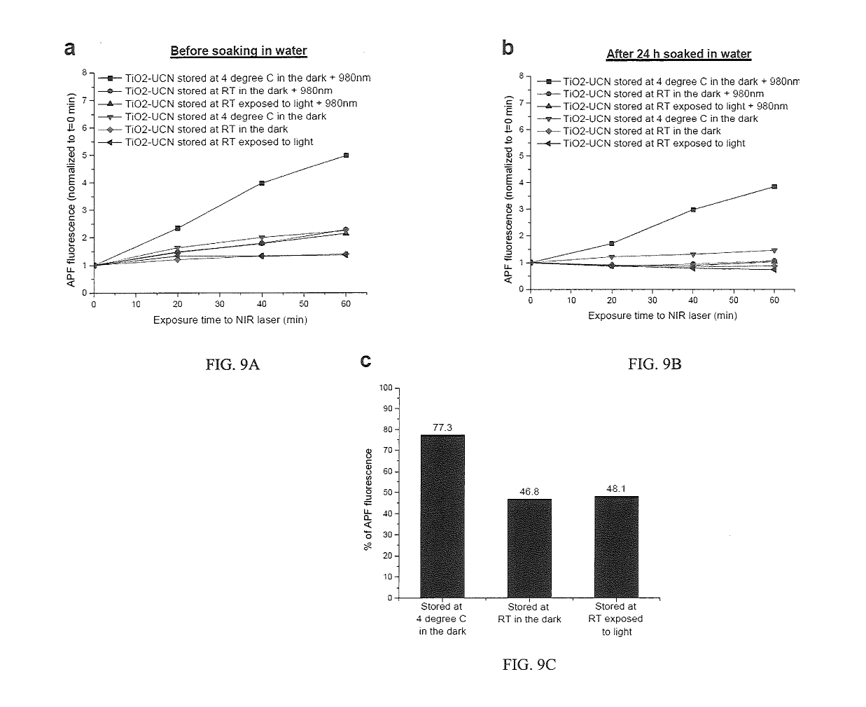

As these nanoparticles are intended for subsequent use as a PDT agent in living cells and organisms that are mainly made up of aqueous solutions, their stability for ROS production when soaked in water for long duration was assessed. Here, the TiO₂—UCN powder was soaked in water at RT for up to 24 h during which their activity for ROS production was assessed at different time points. As evident in FIG. 8, TiO₂—UCN that has been in contact with water showed a gradual decline in their activity for ROS production but this reached a statistical significance only at 24 h of soaking (P=0.04225 at 24 h compared to 0 h of soaking in water). An effort to delay this gradual loss in activity for ROS production from TiO₂—UCN that is in contact with water was attempted by using the approach of storing it at different conditions. TiO₂—UCN powder that has aged for 20 days under different storage conditions of either 4° C. in the dark, RT in the dark or RT but exposed to light (from the previous study indicated as FIG. 5 above and reproduced in this section as FIG. 9A) was soaked in water and the suspension was kept at either 4° C. in the dark, RT in the dark or RT but exposed to light, respectively, for 24 h. After 24 h of storage under these different conditions, their activity for ROS production was assessed (FIG. 9B). It is apparent from FIG. 9C that loss in activity for ROS production from these TiO₂—UCN soaked in water was best retarded when the suspension of nanoparticles was stored at 4° C. in the dark, whereby ROS production dropped to only 77.3% as compared to those stored at RT in the dark or RT but exposed to light wherein the drop in ROS production reached a considerable level of 46.8% and 48.1% respectively. In preserving its activity for ROS production, it is thus best if TiO₂—UCN suspension in aqueous solution be kept on ice while waiting for their administration into cells/animals.

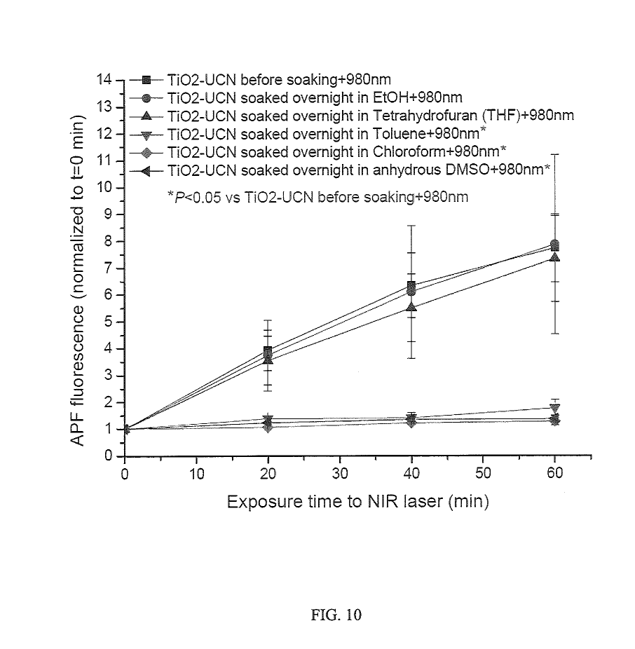

With this realization that TiO₂—UCN slowly loses its activity for ROS production when in contact with water, it now becomes important to search for a solvent that would best preserve it. This is based on the rationale that these nanoparticles would subsequently be subjected to surface modification with molecules such as polyethylene glycol (PEG) and cancer-specific targeting agent (that will help to improve its biocompatibility and targeting efficacy, respectively) in later part of the study. Oftentimes, such surface modification steps would require the nanoparticles to be in contact with a certain solvent for long hours in order for sufficient grafting/conjugation to take place. To investigate which solvent is best in retaining the TiO₂—UCN activity for ROS production, the nanoparticles were soaked overnight in different solvents of either ethanol (EtOH), tetrahydrofuran (THF), toluene, chloroform or anhydrous DMSO. Their stability for ROS production before and after soaking in the different solvents was then compared. While soaking the nanoparticles in toluene, chloroform or anhydrous DMSO brings a sharp drop in their activity for ROS production (P=0.01015, 0.00711 and 0.00746, respectively, compared to before soaking) soaking in EtOH or THF do not seem to have such an effect on the nanoparticles (FIG. 10). In fact, the amount of ROS produced from nanoparticles soaked in either EtOH or THF seemed barely unchanged when compared to the amount of ROS produced before they were soaked in these solvents (P=0.97243 and 0.85389, respectively, compared to before soaking). Therefore, EtOH or THF is most apt to be used as a solvent for subsequent surface modification step.

Unlike existing UCN-based PDT drugs, TiO₂ is coated on the UCN core to form a defined core-shell structure, with the TiO₂ layer serving directly as the photosensitizer drug. In comparison to existing UCN-based PDT drugs, the composite materials and therapeutic compositions of the present invention are better agents of photodynamic therapy. Specifically, previously reported TiO₂—UCN nanocomposite¹⁰⁻¹² shows non-uniformity in size and dispersion as well as irregularity in shape. Furthermore, previously reported particles¹² are in the micrometer range; and are thus correctly classified as microcrystals. Such structures are less useful than nanoparticles in biological applications because they are less easily taken up by cells in the body. In some works¹¹, the TiO₂ semiconductor component is configured as nanoparticles that merely surround the UCN core, rather than forming a continuous layer of TiO₂ coating on the UCN core to give rise to the clearly defined core-shell structure that is presented in this invention. Furthermore, the composite materials demonstrate no problem of leaching out of photosensitizer molecules as the photosensitizer TiO₂ is directly coated on the UCN surface to form a core-shell structure. This is in contrast to photosensitizer-loaded mesoporous-silica-coated UCN¹³⁻¹⁵, in which the photosensitizer molecules are simply adsorbed to the pores of the silica shell. Still further, photoinduced TiO₂ results in the generation of more than one type of reactive oxygen species (ROS) that includes hydroxyl radicals, superoxide anions and hydrogen peroxide. This versatility is compared to photoinduced conventional photosensitizer molecules, such as chlorine 6 and merocyanine 540), that produce only singlet oxygen molecules. Hence, the use of the composite materials of the present invention as agents of photodynamic therapy enables a multi-pronged mechanism of killing target cells, thus allowing the materials to be more effective than conventional photosensitizer molecules.

The composite materials of the present invention have a wide range of possible applications. For example, the materials may be used as compositions in photodynamic therapy for a large range of tumor sizes, types and locations. By having the potential to overcome the limitation of penetration depth in conventional PDT, NIR-PDT using TiO₂—UCN can be used for the treatment of thick, bulky or deep-seated tumors, thereby offering PDT as a treatment option for a larger range of tumor sizes, types and locations. The TiO₂—UCN materials can also be used as detoxifying agents in the remediation of waste water for environmental cleanup through activation of the photo-oxidation of organic pollutants using safe NIR light excitation, in contrast to direct excitation by harmful UV light. The TiO₂—UCN materials also have antimicrobial activity through releasing ROS under safe NIR light excitation (as opposed to direct excitation by harmful UV light) that can be used to sterilize and deodorize surfaces in homes and hospitals. In a further application, TiO₂—UCN can be coated onto glass for development of hydrophilic surfaces activated by UV and visible upconverted light upon excitation by safe NIR light. This can be exploited for the development of self-cleaning glass and anti-fogging coatings. In a further embodiment, radicals generated from NIR light activated TiO₂—UCN in solution or suspension can be used to cleave protein that contains the amino acid proline at the site where proline is present. This may provide an alternative tool that is facile, inexpensive and rapid for researchers to have a highly tunable protein cleavage process, compared to current methods that require proteolytic enzymes or chemical agents and typically a second reagent to discontinue cleavage.

Modification of the surface of the core-shell TiO₂—UCNs with poly-ethylene glycol (PEG) imparted stability and stealth properties, making it more conducive for biological applications, and therefore preventing saturation of the cell-killing ability of the nanoparticle. Specifically, upon exposure of these modified TiO₂—UCNs to NIR both in vitro and in vivo that nanoparticles described in the present invention allow users to treat solid tumors.

Accordingly, in an example embodiment, the present invention is a nanocomposite for photodynamic therapy, comprising: an upconversion nanoparticle, wherein the nanoparticle, upon excitation by near infrared light, emits light of a wavelength from about 330 nm to about 675 nm; and a continuous and uniform outer coating on the outer surface of the nanoparticle, the coating comprising a semiconductor material, wherein the light emitted from the nanoparticle is of a wavelength sufficient to excite one or more electrons from a valence band of the semiconductor material to the conduction band of the semiconductor material, and generates at least one type of reactive oxygen species (ROS).

In another embodiment, the nanoparticle comprises NaYF₄ nanocrystals doped with from about 10 mole % to about 30 mole % Yb³⁺ and from about 0.3 mole % to about 2 mole % Tm³⁺.

In another embodiment, the Y:Yb:Tm molar ratio is 79.5:20:0.5.

In another embodiment, the semiconductor material is TiO₂.

In another embodiment, the nanocomposite further comprises an intermediate coating layer comprising a silica-based composition, wherein the intermediate coating layer is positioned between the upconversion nanoparticle and the outer coating comprising the semiconductor material.

In another embodiment, the intermediate coating layer further comprises 3-aminopropyl-trimethoxysilane.

In another embodiment, the nanocomposite is modified with a targeting agent.

In another embodiment, the targeting agent is linked to the semiconductor surface through a linking group.

In another embodiment, the linking group is poly(ethylene)glycol.

In another embodiment, the targeting agent is an affibody, antibody, aptamer, peptide, or folic acid.

In another example embodiment, the present invention is a composition for photodynamic therapy, comprising: a targeting agent bound to a scaffold comprising a nanocomposite, wherein the nanocomposite comprises: an upconversion nanoparticle, wherein the nanoparticle, upon excitation by near infrared light, emits light of a wavelength from about 330 nm to about 675 nm; and a continuous coating on the outer surface of the nanoparticle, the coating comprising a semiconductor material, wherein the light emitted from the nanoparticle is of a wavelength sufficient to excite one or more electrons from a valence band of the semiconductor material to the conduction band of the semiconductor material, and the semiconductor material, after excitation, is of an energy sufficient to generate at least one reactive oxygen species.

In another embodiment, the targeting agent is linked to the semiconductor surface, optionally through a linking group.

In another example embodiment, the present invention is a method of generating reactive oxygen species, comprising irradiating with near infrared light a sample comprising the nanocomposite of any one of the example embodiments above, and one or more oxygen sources selected from water or oxygen for a period of time sufficient to excite one or more electrons from a valence band of the semiconductor material to the conduction band of the semiconductor material, wherein the one or more oxygen sources undergoes a redox reaction to form a reactive oxygen species.

In another example embodiment, the present invention is a nanocomposite composition, comprising: a plurality of nanocomposites of any of the example embodiments above, wherein the nanocomposites are uniformly distributed throughout the composition, and further wherein the nanocomposites are uniform in shape and size.

In another embodiment, the linking group is a dispersion stabilizer.

In another embodiment, the dispersion stabilizer is a PEG.

In another embodiment, the molecular weight of the dispersion stabilizer is 2000 Da or greater.

In another example embodiment, the present invention is a method of administering photodynamic therapy to treat a biological target in a subject, the method comprising: administering a therapeutically effective amount of a nanocomposite of any of the above embodiments to the subject; exposing the nanocomposite to near infrared light sufficient to cause the nanocomposite particle to emit light of a wavelength of about 330 nm to about 675 nm such that the generated at least one reactive oxygen species treats the biological target. In an example embodiment, the wavelength sufficient to excite one or more electrons from a valence bond of the semiconductor material to a conduction band of the semiconductor material is 980 nm.

In another embodiment, the biological target is a cell surface receptor that is overexpressed in a cancerous cell.

In another embodiment, the cell surface receptor that is overexpressed is an epithelial growth factor receptor.

In another embodiment, the cancer cell is an abnormally proliferating cell of any origin.

In another embodiment, the cancerous cell is an oral squamous cell.

### Construction and Studies of TiO₂—UCNs

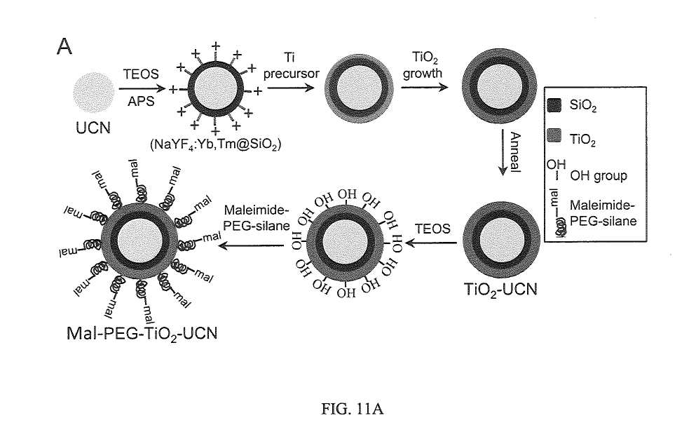

Addition of tetraethyl orthosilicate (TEOS) conferred hydroxyl (OH) groups on the TiO₂ shell for the attachment of silane group of maleimide-PEG-silane, resulting in the formation of Mal-PEG-TiO₂—UCNs. Transmission electron microscopy (TEM) of both TiO₂—UCNs and Mal-PEG-TiO₂—UCNs revealed a uniform spherical shape with a well-defined core-shell structure and average primary particle size of ˜50 nm (FIGS. 11B-C). The average diameter of the UCN core was ˜25 nm, surrounded by a silica layer and TiO₂ shell with a combined thickness of ˜12.5 nm (FIG. 12A). Upon 980 nm NIR light irradiation, TiO₂—UCNs emitted upconverted light in UV, visible and NIR regions of the spectrum (FIG. 11D and FIG. 12B). The UCN's blue emission (peaks at 450 and 475 nm of FIGS. 11D and 12B) could be used to track the nanoparticles uptake by cells, whereas for in vivo imaging its emission at the NIR region (800 nm) could be utilized to capture signals from deeper tissues. Fourier transform infrared (FT-IR) absorption spectra confirmed successful grafting of maleimide-PEG-silane on the surface of TiO₂—UCN, with characteristic peaks of PEG appearing at ˜2885 cm-1 and ˜1470-1350 cm-1 corresponding to C—H stretching and C—H bending respectively (FIG. 11E).

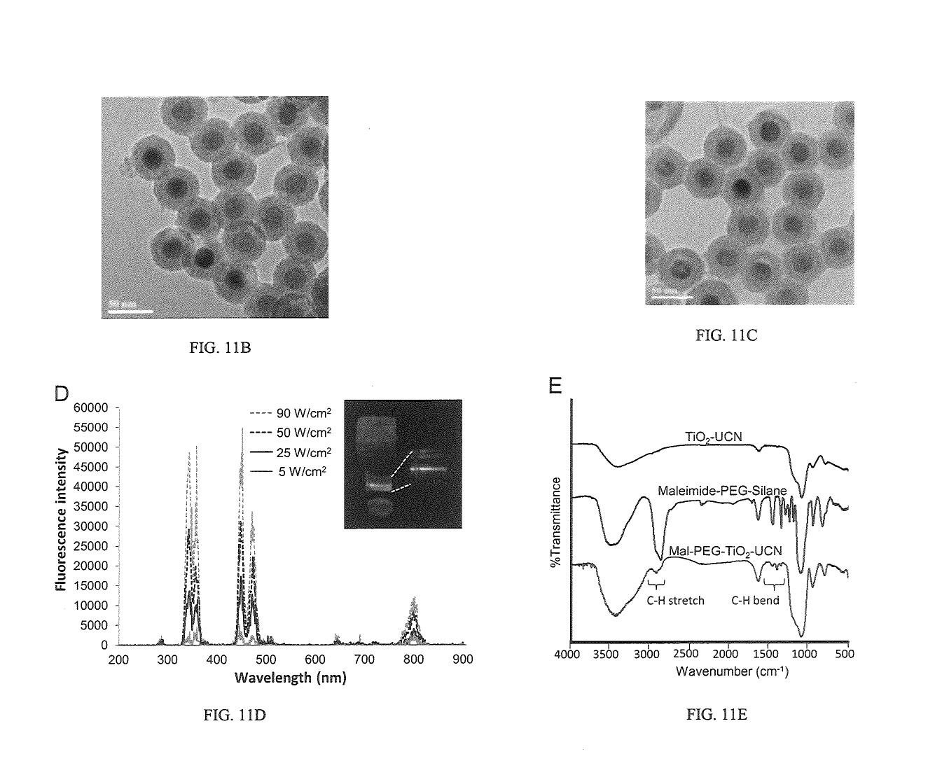

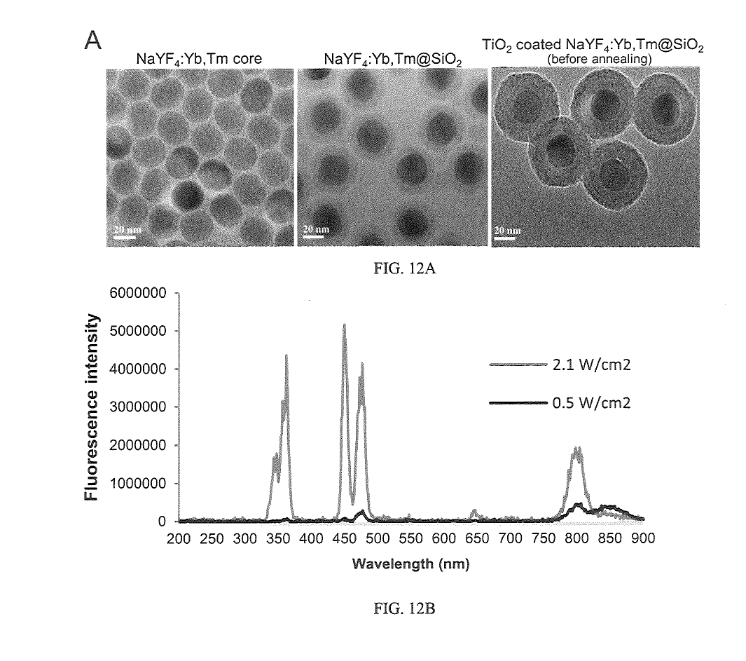

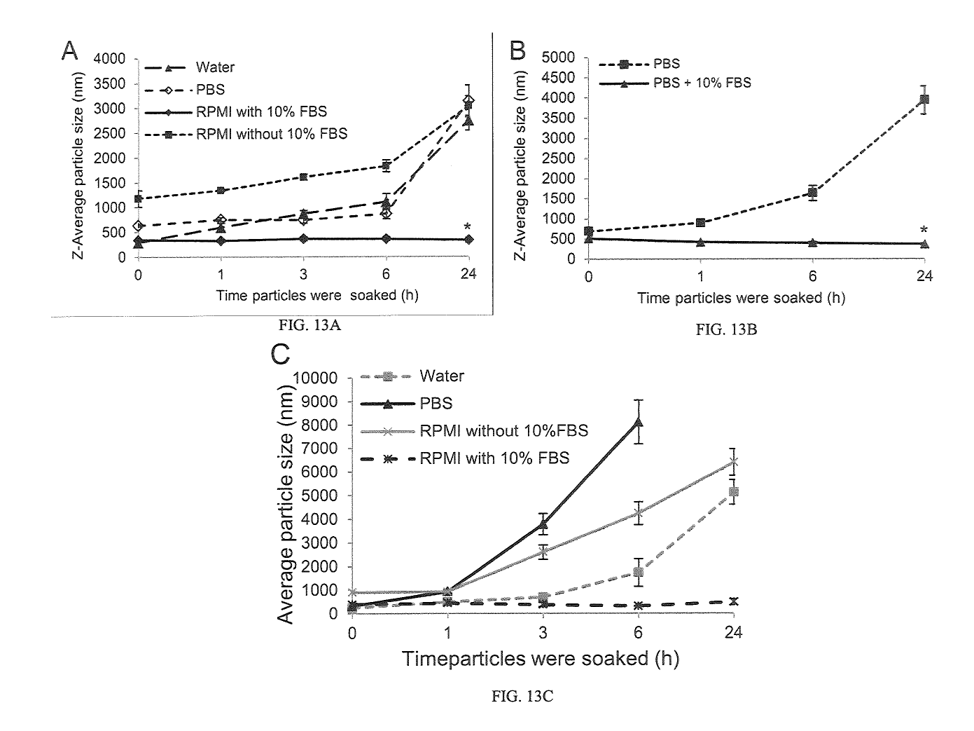

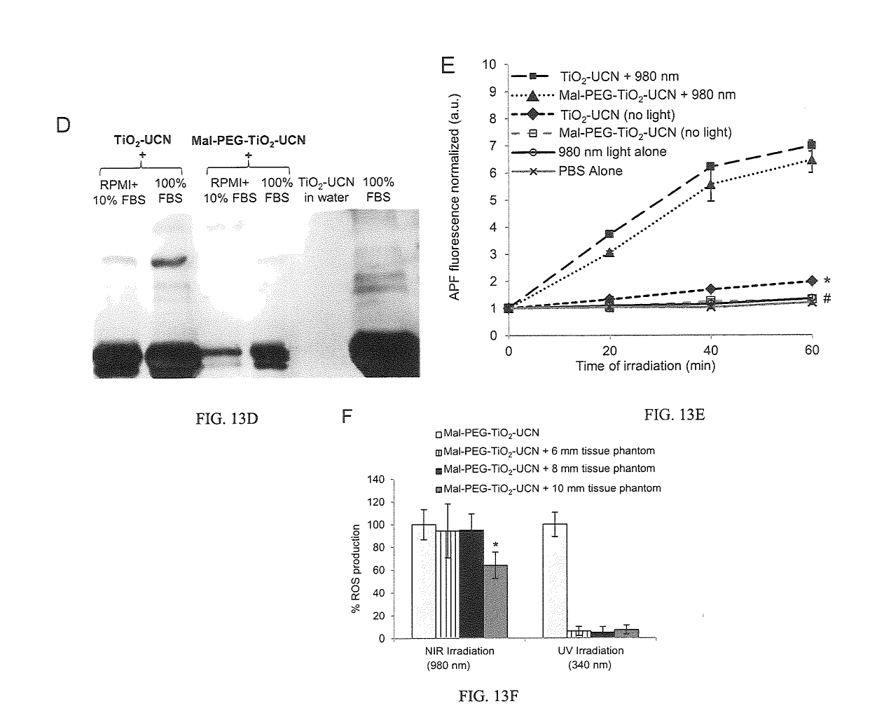

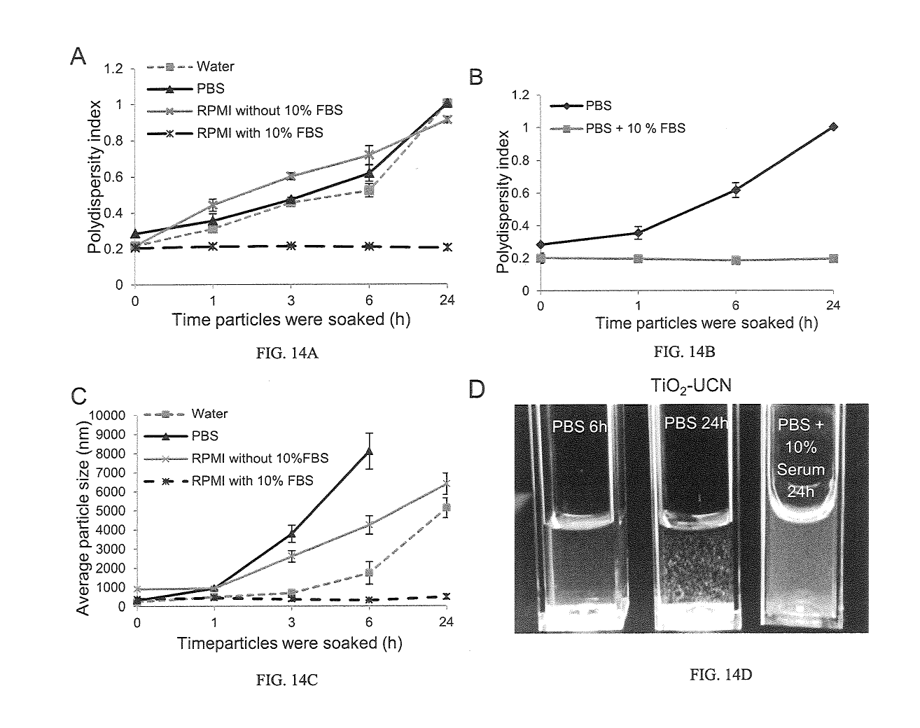

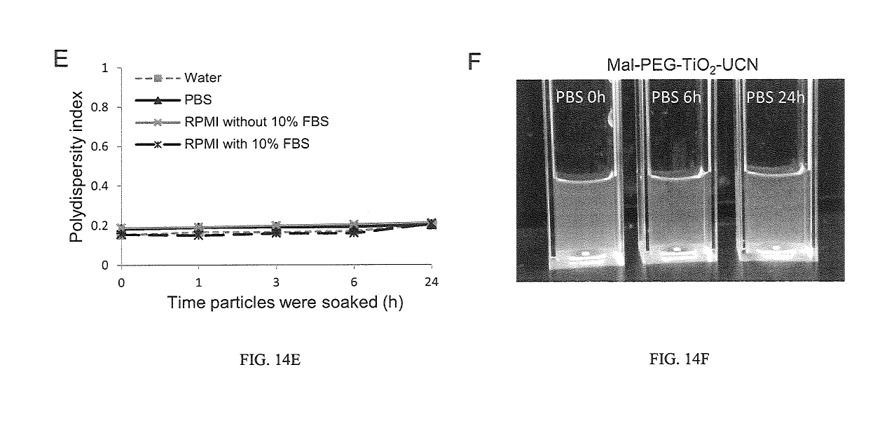

Initial studies using TiO₂—UCNs revealed formation of large aggregates (in the range of micrometers) in water and various physiological solutions at room temperature (RT) (FIGS. 13A and 14 A-B). While it is known that hydrodynamic size of nanoparticles dispersed in liquid is often larger than its primary particles size as measured by TEM, they also increase substantially as a function of time and temperature (FIG. 14C) leading to rapid sedimentation of the aggregates. However, in RPMI-1640 medium (RPMI) with 10% fetal bovine serum (FBS), TiO₂—UCNs formed a stable dispersion. This was compared to the aggregation characteristic of TiO₂—UCNs in phosphate buffered saline (PBS) and PBS with 10% FBS, and found that there was over 10 fold increase in particle size in the absence of FBS (FIG. 13B and FIG. 14D).

TABLE 1

| | TiO2-UCN | | Mal-PEG-TiO2-UCN | |
|---|---|---|---|---|
| | Zeta-Potential | | Zeta-Potential | |
| | (mV) | PDI | (mV) | PDI |
| Water | −25.5 ± 6.8 | 0.216 ± 0.012 | −15.3 ± 5.3 | 0.154 ± 0.004 |
| PBS | −24.0 ± 2.3 | 0.284 ± 0.005 | −8.09 ± 0.5 | 0.183 ± 0.002 |
| RPMI without | −9.6 ± 1.3 | 0.217 ± 0.002 | −8.16 ± 0.3 | 0.188 ± 0.004 |
| FBS | | | | |
| RPMI with | −7.5 ± 0.9 | 0.204 ± 0.005 | −10.4 ± 0.3 | 0.155 ± 0.008 |
| 10% RBS | | | | |

Zeta-potential and polydispersity index (PDI) of 100 μg/ml TiO₂-UCN and Mal-PEG-TiO₂-UCNs, immediately after dispersing in various solutions.

The surface charge of these nanoparticles were studied by measuring the zeta-potential. When TiO₂ nanoparticles are dispersed in water, the surface of the nanoparticle is generally covered by hydroxyl group (Ti^{IV}+H₂O→Ti^{IV}—OH+H⁺)¹⁶, which imparts a negative charge to it. Although TiO₂—UCNs had a negative zeta-potential (Table 1), its value was greater than −30 mV. Generally, zeta-potential of more than +30 mV or less than −30 mV is required to provide enough repulsive forces to counterweigh the van der Waals force of attraction that leads to particle aggregation. Presumably, the low zeta potential (in the range of −25.5±6.8), could be the reason for the observed increase in the hydrodynamic sizes of TiO₂—UCNs in water. On the other hand, in a high ionic strength dispersing media (like PBS and RPMI), the hydrodynamic size of nanoparticles are affected by both the zeta potential and electrical double layer thickness. It is well known that electrical double layer thickness around the nanoparticles becomes smaller when dispersed in high ionic strength solutions,¹⁷ which correspondingly leads to a weaker electrostatic repulsive force resulting in large sized aggregates. It is worth noting that the zeta-potential of TiO₂—UCNs in PBS is in the range of −24.0±2.3 and that in RPMI without FBS is −9.6±1.3.

Thus, both a weak zeta-potential and smaller electrical double layer, could have resulted in the formation of large aggregates in PBS and RPMI. It was then found that binding of serum proteins to the surface of TiO₂—UCNs forming a “protein corona”, helped to maintain dispersion stability.¹⁸ The presence of proteins on the surface of TiO₂—UCNs creates a physical steric barrier, preventing the nanoparticles from approaching one another.¹⁹ Although serum protein binding might momentarily seem to solve the problem of aggregation, it has implications as certain components of the protein corona may act as opsonins.^{20,21} Opsonization can eventually lead to the recognition and removal of these nanoparticles from circulation by the macrophages of the mononuclear phagocytic system, leading to decreased bio-availability during their application in vivo. Thus, modifying the surface of TiO₂—UCNs is desirable to improve its dispersion stability, as well as to prevent the attachment of opsonins. PEGylation, constitutes the most efficient and widely used anti-opsonization and steric stabilization strategy.²² Typically, a PEG chain with molecular weight of 2000 Da or greater is required to achieve stealth characteristics,²³ such that it remains invisible to the phagocytic cells.

Hence, a maleimide-PEG-silane with a molecular weight of 2000 Da was chosen to surface-modify TiO₂—UCNs. The maleimide group will serve as a reactive functional group for further conjugation of tumor targeting moieties, for targeted delivery of nanoparticles in the future. PEGylation of nanoparticles is known to decrease its surface energy and minimizes the van der Waals force of attraction between the nanoparticles, by increasing the steric distance between them, resulting in stable nanoparticle dispersion.²⁴ PEGylation of TiO₂—UCNs conferred dispersion stability up to 24 h, with smaller hydrodynamic sizes (˜300 nm), even in the absence of FBS (FIG. 13C and FIGS. 14E-F). As PEGylation further reduced the negative zeta-potential of TiO₂—UCNs (Table 1), the dispersion stability observed in the case of Mal-PEG-TiO₂—UCNs is assured not by the electrostatic repulsion but by the steric repulsion between the PEG chains. Furthermore, while the polydispersity index (PDI) of TiO₂—UCN was slightly greater than 0.2 in the different solutions except in RPMI with 10% FBS, PDI of Mal-PEG-TiO₂—UCN was less than 0.2, again indicating formation of a stable dispersion (Table 1).

The ability of PEGylation to reduce protein adsorption was studied by soaking nanoparticles in RPMI with 10% and 100% FBS and then centrifuging to separate out the nanoparticles with attached serum proteins. Gel electrophoresis and silver staining revealed that PEGylation significantly reduced adsorption of serum proteins to the surface of TiO₂—UCNs (FIG. 13D). Upon NIR irradiation, both TiO₂—UCNs and Mal-PEG-TiO₂—UCNs generated significant amount of ROS in PBS compared to the corresponding non-irradiated nanoparticles (FIG. 13E). In addition to this, it was determined that the tissue penetration ability of NIR light as well as its ROS production ability following indirect excitation of TiO₂ shell by the upconverted UV light and further compared it with direct excitation of TiO₂ shell with UV light, by utilizing tissue phantoms of varying thickness (ranging from 6-10 mm) (FIG. 13F). It was found that while there was only about 36% drop in ROS production when irradiated with NIR light in the presence of a 10 mm tissue phantom, there was over 90% drop in ROS generation when irradiated with UV light. Thus, when compared to direct excitation of TiO₂ shell with UV light, indirect excitation with NIR light has the advantage of penetrating thick tissues, which further highlights the suitability of our nanoconstruct in the treatment of solid or deep-seared tumors.

### Uptake of Nanoparticles in Macrophage and Cancer Cells

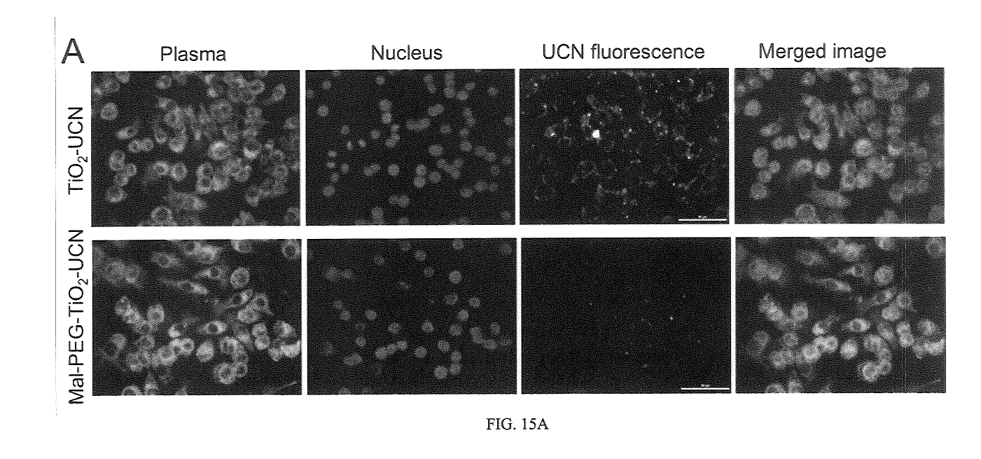

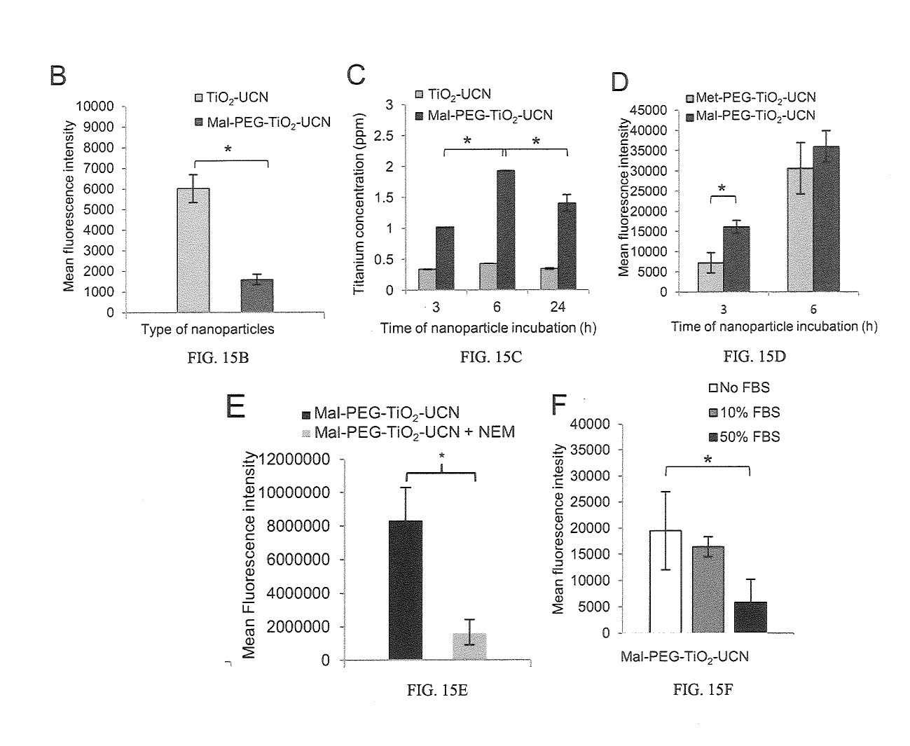

The presence of PEG also reduced the recognition and uptake of nanoparticles by mouse macrophage cells. TiO₂—UCNs were taken up about 4 times more than Mal-PEG-TiO₂—UCNs after 1 h of incubation with macrophage cells (FIGS. 15A-B).

Both TiO₂—UCNs and Mal-PEG-TiO₂—UCNs were incubated with human oral squamous cell carcinoma (OSCC) cells for different time-points, and washed to remove the unbound nanoparticles. Subsequently, the cells were digested and the lysate was quantitatively analyzed for titanium content by inductively coupled plasma atomic emission spectroscopy (ICP-AES). It was found that while PEGylation reduced uptake of nanoparticles by macrophage cells, it significantly enhanced the uptake of nanoparticles into OSCC cells (FIG. 15C) compared to TiO₂—UCNs. Moreover, there was significantly greater uptake at 6 h post incubation compared to 3 h incubation. At the end of 6 h incubation, the nanoparticles were mostly observed in the cytoplasm. However, following 24 h incubation there was a drop in titanium content compared to the 6 h time point. Thereafter, some of the ingested nanoparticles could have been exocytosed by 24 h.^{25,26}

It is often argued that while PEGylation reduced macrophage recognition and uptake, it could in turn lead to reduced cellular uptake, decreasing the therapeutic potential of such nano-delivery systems. However, an increased uptake of Mal-PEG-TiO₂—UCNs by the cancer cells compared to TiO₂—UCNs was observed. As maleimide group rapidly and specifically binds to the thiol group, it is possible that PEG-maleimide-modified nanoparticles could target cell surface thiols, resulting in their enhanced cellular internalization.²⁷ PEG-silane was utilized with (maleimide-PEG-silane) and without malemide group (methoxy-PEG-silane, 2000 Da) to surface modify TiO₂—UCNs, and compare cell-binding and internalization efficiency in OSCC cells. The results revealed a significant increase in the uptake of Mal-PEG-TiO₂—UCNs into the OSCC cells as early as 3 h (FIG. 15D). When cell surface thiols were pre-blocked with N-ethylmaleimide (NEM), a 5 fold reduction was observed in the internalization of Mal-PEG-TiO₂—UCNs in cancer cells (FIG. 15E). The uptake of Mal-PEG-TiO₂—UCNs was not significantly affected by the presence of 10% FBS compared to the uptake in medium without FBS, but dropped about 4 folds in the presence of 50% FBS (FIG. 15F).

### Dark-Toxicity of Nanoparticles

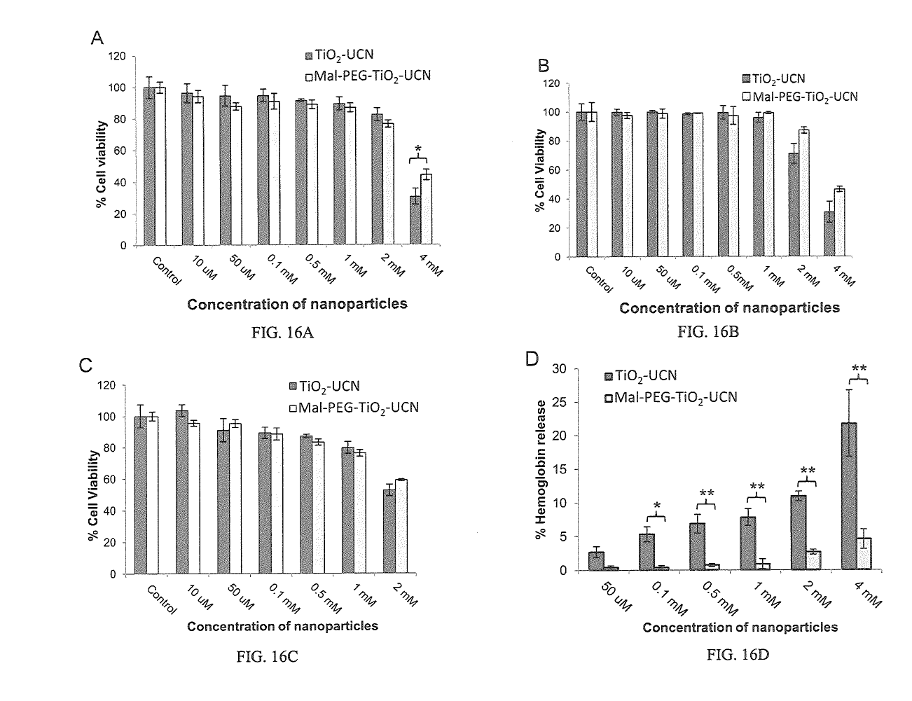

Human OSCC cells can serve as a model for both in vitro and in vivo studies as these cells overexpressed epithelial growth factor receptors on its cell surface,²⁸ which can be utilized to specifically target the developed nanoparticles to these cancer cells. There is no significant difference between cell-viability of untreated OSCC cells and cells treated with TiO₂—UCNs or Mal-PEG-TiO₂—UCN up to a concentration of 1 mM (FIG. 16A). At 2 mM, though the cell viability is still above 80%, it is significantly lower than the viability of untreated control cell and drops below 50% at very high concentration of 4 mM. Again, there was no significant difference between the toxicity of unmodified and surface modified TiO₂—UCNs except at high concentration above 2 mM, where the PEGylated TiO₂—UCNs seemed to be significantly less toxic than the unmodified version (P=0.0082). Although, the cell uptake and dark-toxicity of OSCC cells cannot be compared with normal human fibroblast (NHF) cells, owing to the difference in cell sizes and growth patterns, a similar trend in dark-toxicity was also observed when the nanoparticles were incubated with NHF cells (FIG. 16B). As the standard MTS ([3-(4,5-dimethylthiazol-2-yl)-5-(3-carboxymethoxyphenyl)-2-(4-sulfophenyl)-2H-tetrazolium) proliferation assay only reflects the mitochondrial enzyme activity, the dark-toxicity of the nanoparticles was also evaluated using the trypan-blue dye exclusion method which is based on the fact that dead cells lose their membrane integrity and become permeable to the dye. There was no significant difference between cell-viability of untreated OSCC cells and cells treated with TiO₂—UCNs or Mal-PEG-TiO₂—UCN up to a concentration of 1 mM and cell-viability remained above 80% (FIG. 16C). Thereafter, it reduced to 60% at a concentration of 2 mM. Hence, for all subsequent in vitro work, the maximum concentration of nanoparticles used was fixed at 1 mM.

### Hemolysis Assay

The hemocompatibility of the nanoparticles at a concentration range of 50 μM-4 mM were evaluated with red blood cell (RBC) lysis assay. As shown in FIG. 16D, both TiO₂—UCNs and Mal-PEG-TiO₂—UCNs displayed a dose-dependent increase in hemoglobin release. However, the RBC lysis caused by TiO₂—UCNs at concentrations greater than 50 μM were significantly greater than Mal-PEG-TiO₂—UCNs. Percent hemolysis levels in 4 mM TiO₂—UCN treated RBCs were 21.7%. On the other hand, the hemoglobin release caused by Mal-PEG-TiO₂—UCNs was well below 5% across the range of concentrations investigated. This indicates that PEG modified TiO₂—UCNs exhibit excellent hemocompatibility and is suitable for in vivo application.

### In Vitro PDT and Cell Death

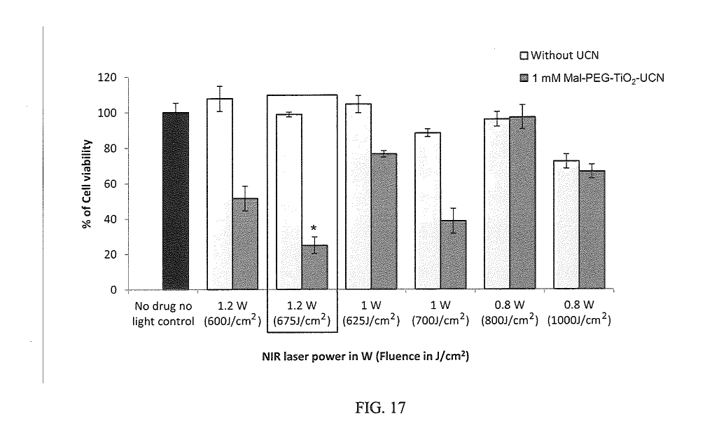

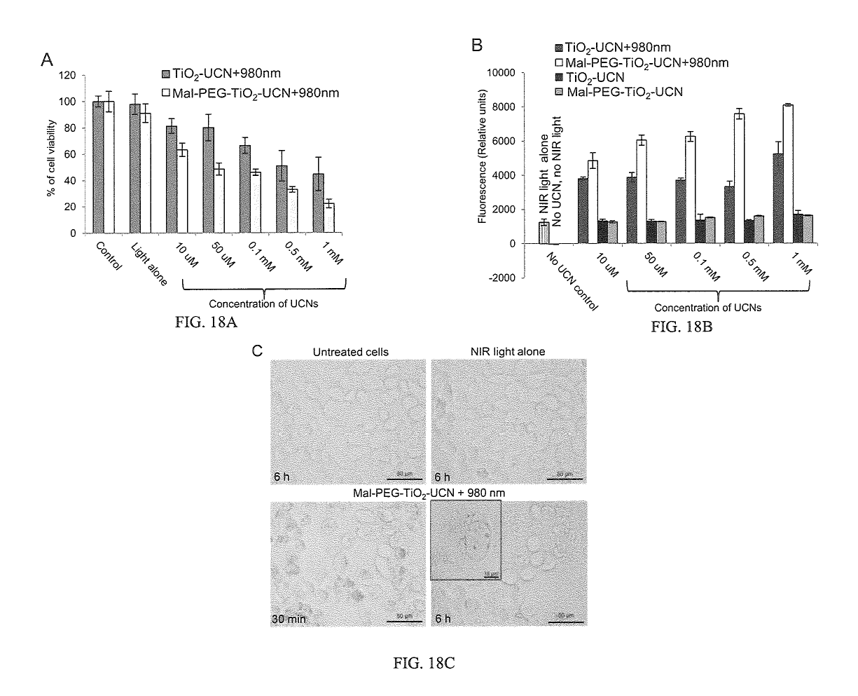

Before evaluating the efficiency of the synthesized nanoparticles for PDT, it is essential to optimize the PDT parameters like the light dose and time of irradiation such that the NIR light itself does not kill the cells. To achieve this, OSCC cells were subjected to a range of NIR laser dose at 980 nm, to determine a dosage that can be well tolerated by the cell, but is detrimental to the cells in the presence of nanoparticles. Since, here the photocatalyst TiO₂ is excited indirectly by an upconverted UV light (anti-Stokes scheme) and not by direct excitation with UV as in a typical PDT regime, the excitation power density that is required will be relatively higher due to the low efficiency of the upconversion process.²⁹ It was found that a NIR laser power of 1.2 W under continuous irradiation for 5 min 20 sec (at power density of ˜2.1 W/cm²) delivering a light fluence of 675 J/cm² was well tolerated by OSCC cells (FIG. 17), with >95% cell viability. At the same time, in the presence of 1 mM Mal-PEG-TiO₂—UCNs, irradiation using this optimized light dose killed about 80% of the cells. Any further decrease in laser power and increase in time of irradiation did not hit such a right balance, leading to the assumption that there exists a minimum threshold excitation power density required by the UCN core, below which it cannot actively upconvert and excite the TiO₂ in the shell to produce sufficient ROS for effective cell killing. On the other hand, a further increase in excitation power density may lead to thermal decline of the cells. In vitro PDT on OSCC cells using the optimized light parameters, showed that Mal-PEG-TiO₂—UCNs produced significantly more cell death (78%) compared to TiO₂—UCNs (56%) at a concentration of 1 mM (FIG. 18A). Thus, surface modification of TiO₂—UCNs significantly improved the PDT efficacy of TiO₂—UCNs in vitro.

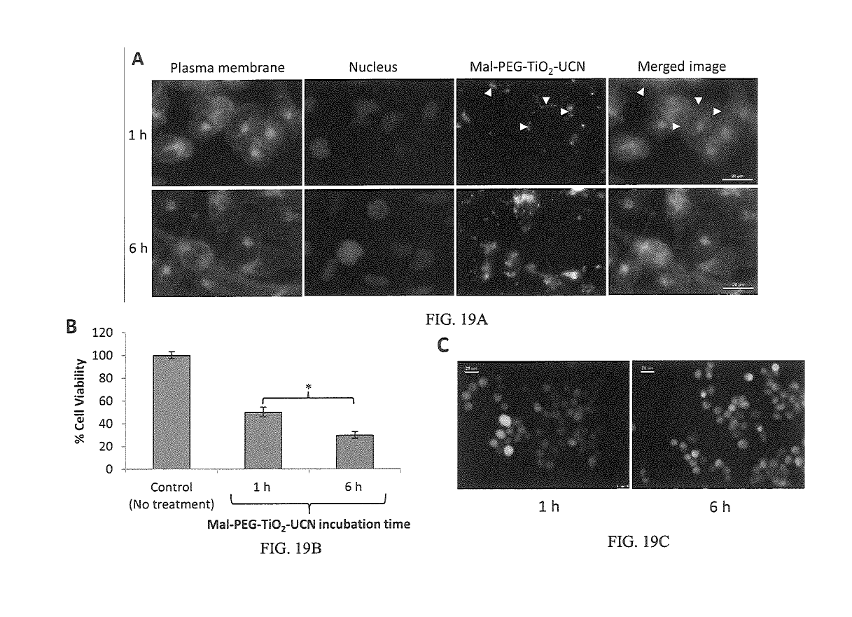

As a further proof that the cell-death is indeed bought about by ROS that is generated by the photoactivation of TiO₂ shell on the UCNs, the ROS generation was quantitatively evaluated within 30 min after irradiation of the cells. At a concentration of 1 mM, the Mal-PEG-TiO₂—UCNs in the presence of 980 nm light produces significant amount of ROS compared to TiO₂—UCNs, resulting in better PDT efficacy probably due to the higher uptake of the nanoparticles by the cancer cells (FIG. 18B). Additionally, in the case of Mal-PEG-TiO₂—UCN, there was a considerable dose-dependence in the production of ROS. Furthermore, it was found that when Mal-PEG-TiO₂—UCNs were incubated for a shorter duration of 1 h, the particles were mostly seen attaching to the cell membrane, with very minimal uptake of the particles into the cells (FIGS. 19A-C). However, after 6 h of incubation the particles were mostly seen inside the cell, predominantly in the cytoplasm. On comparing the cell-viability, it was observed that there was about 50% cell death following PDT after a mere 1 h incubation of Mal-PEG-TiO₂—UCN. Nevertheless, cell death was more pronounced (>70%) following PDT after 6 h incubation. This observation is consistent with in vitro nanoparticle uptake data, that there is a higher uptake of nanoparticles after a 6 h incubation period, due to which there was a greater cell-kill following PDT.

The discussion below is a theory regarding the mechanism of cell death using the nanoparticles of the present invention and is not intended to be limiting. PDT is known to induce cell-death by apoptosis, necrosis or autophagy depending on the cell type, the nature and localization of the PSs and the light dose. When untreated OSCC cells and cells irradiated with NIR light alone were stained with trypan blue, very few cells seemed to take up the blue dye within 30 min of light irradiation (FIG. 18C). However, when the cells were incubated with Mal-PEG-TiO₂—UCNs for 6 h and then irradiated with NIR light, majority of the cells stained blue within 30 min of treatment, indicating loss of membrane integrity although the cells appeared to maintain its shape. Six hours after NIR light irradiation, there was cell membrane bleeding and complete rupture of cell membrane suggesting severe cellular insult via necrotic pathway of cell death.

Controlling the amount of PS loaded in UCN constructs and achieving stable loading of sufficient amount of PS, has been one of the major bottleneck in the design of UCN based PDT nanoplatforms impeding its translation to even distantly comparable advances in the clinics. The methods and compositions of the present invention ensures the formation of a well-defined core-shell structured nanoconstruct in which a single mono-disperse UCN core is surrounded by a thin layer of TiO₂, the amount of which can be precisely controlled. The in vitro results clearly indicate the potential application of this biocompatible nanoconstruct in NIR-triggered deep-tissue PDT.

### Examples

### Example 1

### Synthesis of Upconversion Nanoparticles

NaYF₄:20% Yb, 0.5% Tm nanocrystals were synthesized as follows: YCl₃ (0.8 mmol), YbCl₃ (0.2 mmol) and TmCl₃ (0.005 mmol) were mixed with 6 mL oleic acid and 15 mL octadecene (ODE) in a 50 mL flask. The solution was heated to 160° C. to form a homogeneous solution, and then cooled down to room temperature. 10 mL of methanol solution containing NaOH (2.5 mmol) and NH₄F (4 mmol) was slowly added into the flask and stirred for 30 minutes. Subsequently, the solution was slowly heated to remove methanol, degassed at 100° C. for 10 minutes, and then heated to 300° C. and maintained for 1 h under Argon protection. After the solution was cooled naturally, nanocrystals were precipitated from the solution with ethanol, and washed with ethanol/water (1:1 v/v) for three times. 0.1 mL CO-520, 6 mL cyclohexane and 4 mL 0.01 M NaYF₄ nanosphere solution in cyclohexane were mixed and stirred for 10 min. Then 0.4 mL IGEPAL CO-520 (Polyoxyethylene (5) nonylphenylether, branched) and 0.08 mL ammonia (wt 30%) were added and the container was sealed and sonicated for 20 min until a transparent emulsion was formed. 0.04 mL tetraethylorthosilicate (TEOS) was then added into the solution, and the solution was rotated for two days at a speed of 600 rpm. NaYF₄@SiO₂ nanospheres were precipitated by adding acetone, and the nanospheres were washed with ethanol/water (1:1 v/v) twice and then stored in water.

### Example 2

### Coating of TiO₂ on Upconversion Nanoparticles

For further coating of an amorphous TiO₂ layer, the silica surface was modified with amino groups through grafting (3-aminopropyl)-trimethoxysilane (APS) on the NaYF₄@SiO₂ nanoparticles. In a typical synthesis of NaYN@TiO₂ nanostructures, 0.02 mmol NaYF₄@SiO₂ nanoparticles was dispersed in 10 mL isopropanol (IPA), 0.3 mL ammonia (28 wt %) and 2.5 mL water. Then, 2 mL of titanium diisopropoxide bis(acetylacetonate) solution (0.001 M in isopropanol) was slowly added into the above solution and stirred for 24 h at room temperature (20° C.). Amorphous TiO₂ coated nanoparticles were then collected by centrifugation and washed with IPA solution twice. To achieve a crystallized TiO₂ shell, the NaYF₄@TiO₂ nanoparticles were treated in ethanol in a sealed autoclave at 180° C. for 24 h under an air atmosphere.

### Example 3

### Fluorescence Spectrophotometry

Fluorescence spectra of the nanoparticles was measured with a SpectroPro 2150i spectrophotometer (Roper Scientific Acton Research, MA) equipped with a 1200 g mm⁻¹ grating and a continuous wave (CW) 980 nm diode laser. Nanoparticles were resuspended in the respective solution of either water, phosphate buffered saline (PBS) or Dulbecco's Modified Eagle Medium (DMEM) culture medium with or without 10% fetal bovine serum supplementation, for spectrophotometer measurement.

Surface modification of TiO₂—UCNs with Maleimide-PEG-silane. 4 mg maleimide-PEG-silane (Nanocs Inc., New York, USA) was dissolved in 4 ml of water, to which 4 mg of TiO₂—UCN dispersed in 4 ml ethanol was added. Subsequently, 10 μl of TEOS was added and the solution was stirred at RT for 30 min. At the end of stirring, 150 ul of ammonia (28 wt %) was added drop wise to the solution and stirred for another 3 h at RT. Mal-PEG-TiO₂—UCNs were then collected by centrifuging solution at 8000 rpm for 10 min at 10° C., washed twice with ethanol and then stored at 4° C. For comparison, PEG-silane without maleimide group (methoxy-PEG-silane −2000 Da, Nanocs Inc., New York, USA) was also used to surface modify TiO₂—UCNs using the same protocol.

Characterization of synthesized nanoparticles. Size and morphology of the synthesized nanoparticles were characterized using a JEOL 2010 TEM operating at an acceleration voltage of 200 kV. Fluorescence spectra was recorded on a SpectroPro 2150i spectrophotometer (Roper Scientific Acton Research, MA) equipped with a 1200 g/mm grating and 980-nm VA-II diode pumped solid-state (DPSS) laser. FT-IR spectra were recorded on a Shimadzu IRPrestige-21 model spectrometer (Shimadzu Corporation, Kyoto, Japan). Dynamic light scattering was conducted with the Zetasizer (Nano ZS, Malvern Instruments Ltd., UK) to measure the hydrodynamic diameter, PDI and zeta-potential. Nanoparticles at a concentration of 1 mg/ml in deionized water were sonicated for 20 min before further diluting (100 μg/ml) it in water, PBS, RPMI and RPMI with 10% FBS to determine the average aggregate size with time.

Measurement of ROS production in solution. To measure the ROS generation ability of the unmodified and modified TiO₂—UCNs, aminophenyl fluorescein (APF) (Molecular Probes, Inc., USA) was used as an indicator. UCNs at a concentration of 1 mg/ml in PBS were sonicated for 20 min and APF dye at a final concentration of 10 μM was added to the UCN suspension. The fluorescence of suspension was measured before irradiation at 515 nm by a UV-Vis spectrophotometer (Photonitech, Singapore) under excitation at 490 nm, which is denoted as fluorescence intensity at time 0 h (t=0). The suspension was then irradiated using 980 nm NIR light at a power of 1.2 W for up to 60 min, measuring the fluorescence at every 20 min of irradiation. As the amount of generated ROS is proportional to the fluorescence intensity of APF, the fluorescence intensity is plotted as a function of exposure time.

To demonstrate the tissue penetration abilities, the same experiment was performed by placing tissue phantoms of varying thickness (6-10 mm), in the path of the incident NIR or UV light. Briefly, the tissue phantoms were prepared using 0.5% (w/v) ultrapure agarose, (Invitrogen), 1% (v/v) of intralipid-10% (Kabivitrum Inc.) as the scatterer and 0.1% (v/v) Nigrosin, as the absorber. The tissue penetration and ROS generation abilities of NIR and UV light was expressed as percentage drop in ROS generation from 1 mg/ml Mal-PEG-TiO₂—UCNs following irradiation in the presence of tissue phantoms as compared to direct irradiation of the sample with NIR or UV light without the tissue phantom.

Gel electrophoresis and silver staining. The nanoparticles (TiO₂—UCNs and Mal-PEG-TiO₂—UCNs) at a concentration of 1 mg/ml were treated in RPMI with 10% FBS or 100% FBS for 24 h. The suspension was then carefully layered over 10% glycerol and centrifuged at 15000 rpm for 15 mins. The pellet was collected and resuspended in 200 μl of deionized water. Equal volume of this sample was treated with 2× Laemmli sample buffer (Bio-Rad, USA) and heated for 5 min at 95° C. to reduce the di-sulfide bonds. The samples were then loaded on a 5% Sodium dodecyl sulfate-polyacrylamide gel electrophoresis (SDS-PAGE) gel to separate SDS-denatured proteins at 120 V for 2.5 h. The protein bands were silver stained using the Pierce Silver Stain Kit (Thermo Scientific, USA), following manufacturer's instructions.

Cell-lines. OSCC (CAL-27), mouse leukemic monocyte macrophage cell line (RAW 264.7) and NHF cells (IMR-90) were purchased from American Type Culture Collection (ATCC, USA). The cells were cultured in RPMI-1640 medium (OSCC cells) and Dulbecco's modified Eagle's medium (DMEM) (Macrophages and NHF cells). The media were supplemented with 10% FBS and 1% penicillin-streptomycin. Cells were maintained at 37° C. in a humidified atmosphere containing 5% CO₂.

In vitro macrophage uptake assay. RAW 264.7 mouse macrophages were seeded in S-well chambered slide at a cell density of 25×103 cells per well and incubated overnight to allow the cells to adhere to the floor of the wells. The medium in the wells were replaced with a nanoparticle suspension (TiO₂—UCN or Mal-PEG-TiO₂—UCN) at a concentration of 1 mM (270 μg/ml) in DMEM supplemented with 10% FBS and incubated for 1 h at 37° C. The macrophage cells were then rinsed thrice with 1×PBS to wash away the excess non-ingested nanoparticles and fixed in ice-cold methanol for 10 min. The plasma membrane was stained with Wheat Germ Agglutinin, Alexa Fluor® 488 Conjugate (Molecular Probes, Inc., USA) at a concentration of 5 μg/ml for 10 min. The nucleus was further counterstained with propidium iodide (Molecular Probes, Inc., USA) at a concentration of 500 nM for 5 mins. The cell were gently washed thrice with PBS and mounted using Vectashield mounting medium (Vector Laboratories, CA, USA). The uptake of unmodified and modified TiO₂—UCNs by the macrophages was imaged using an upright Nikon 80i Fluorescence Microscope (Nikon, Tokyo, Japan) equipped with a 980 nm Laser Wide-field Fluorescence add-on (EINST Technology Pte Ltd, Singapore) using a 20× objective (200× magnification). The plasma membrane and nuclei of the cells were visualized under excitation with Hg arc lamp and a standard FITC and TRITC filter set respectively. The uptake of unmodified and modified TiO₂—UCNs by macrophages was also compared by measuring the total fluorescence intensities of UCNs using the Image J 1.47v software (National institute of Health, USA).

In vitro dark-toxicity measurement. OSCC and NHF cells were seeded at a cell density of 8×103 per well in a 96-well plate and incubated overnight to allow it to adhere to the bottom of the plate. The nanoparticles (TiO₂—UCNs or Mal-PEG-TiO₂—UCNs) were prepared at a concentration of 1 mg/ml in sterile PBS, sonicated for 20 min and then diluted in RPMI with 10% FBS at varying concentrations ranging from 10 μM (2.7 ug/ml) to 4 mM (1.08 mg/ml) before adding to the cells. Cells were further incubated for 6 h at 37° C. after which they were gently washed 3 times with 1×PBS to remove the nanoparticles and replaced with fresh culture media. Following 24 h incubation at 37° C., the number of viable cells was determined by MTS assay using CellTiter 96® AQueous One Solution Cell Proliferation Assay (Promega, Madison, Wis., USA) kit as per manufacturer's instructions. The percent cell viability values are reported relative to those of untreated control cells.

For trypan blue staining, 8×104 OSCC cells were seeded in a 12 well plate and treated with nanoparticles as mentioned above. The cells in each of the wells were harvested and collected by centrifugation at 1200 rpm for 5 min. The cells were then stained with 0.4% trypan blue solution in PBS for 5 min before counting using a dual-chamber hemocytometer and a light microscope. Total number of cells and dead (blue coloured) cells were recorded, and the means of three independent cell counts were pooled for analysis. The percentage of viable cells was determined by according to the following formula. Percent of viable cells=100×[(Total number of cells−Number of dead cells)/(Average number cell in the control untreated well)].

Hemolysis assay. Female balb/c nude mice, 6-8 weeks of age, weighing an average of 17 g were obtained from BioLasco, Taiwan. Fresh blood ml) was obtained from mice via cardiac puncture. All procedures carried out in this study were approved by the Institutional Animal Care and Use Committee (IACUC), SingHealth, Singapore and were conducted in accordance with international standards. Red blood cells (RBCs) were separated from plasma by centrifuging at 1500 rpm for 15 min at 4° C. The isolated RBCs were further washed three times with sterile PBS by centrifugation until the supernatant was clear, and resuspended in 2 ml PBS. Then 100 μl of the nanoparticle (both TiO₂—UCN and Mal-PEG-TiO₂—UCN) suspension in PBS at concentrations ranging from 50 μM to 4 mM were added to 100 μl of the RBCs suspension. Following a 2 h at 37° C. under constant shaking, the suspensions were centrifuged at 1500 rpm for 15 min. Subsequently, 100 μl of supernatant from each centrifuge tube was used to analyze hemoglobin release by microplate reader at the wavelength of 576 nm. Control experiments were performed under the same experimental conditions, where 100 μl of the RBCs suspension was added to 100 μl of PBS as a negative control and to 100 μl of 0.5% Triton X-100 as a positive control. The percentage hemolysis was calculated using the following equation:

Hemolysis (%)=(OD576 sample−OD576 negative control)/(OD576 positive control−OD576 negative control)×100%

In vitro uptake of nanoparticles. OSCC cells were seeded in a 145 cm2 cell-culture dish at a density of 3×106 cells per dish and incubated at 37° C. overnight. Cells were treated with UCNs at a concentration of 1 mM for 3, 6 or 24 h; following which the culture medium containing non-internalized nanoparticles were discarded, and the cells were washed three times with phosphate-buffered saline. The cells were then harvested and the intracellular uptake of nanoparticles was determined by measuring the titanium content using ICP-AES.

For imaging uptake of nanoparticles, OSCC and NHF cells were seeded in 8 well chambered slide and incubated overnight; following which the cells were treated with Mal-PEG-TiO₂—UCNs at a concentration of 1 mM for 6 h. The cells were then fixed with ice-cold methanol for 10 mins; plasma membrane and nuclei were stained with Wheat Germ Agglutinin, Alexa Fluor® 488 Conjugate and propidium iodide, washed, coverslipped and imaged using a Nikon 80i Fluorescence Microscope (Nikon, Tokyo, Japan) using a 40× objective (400× magnification) under excitation with Hg arc lamp and a standard FITC, TRITC and DAPI filter set. Furthermore, the uptake of Mal-PEG-TiO₂—UCNs and Met-PEG-TiO₂—UCNs in OSCC cells at various incubation conditions was quantified by measuring the total fluorescence intensities of UCNs using the Image J 1.47v software (National institute of Health, USA). To check the influence of NEM on the cellular uptake of Mal-PEG-TiO₂—UCNs, OSCC cells were pre-blocked with 1 nM NEM in serum free RPMI for 15 min, followed by 6 hr incubation with 1 mM Mal-PEG-TiO₂—UCNs.

In vitro PDT. OSCC cells were seeded into 96-well cell culture plate at a cell density of 8×10³ cells per well. Following overnight incubation at 37° C., the cell were treated with various concentrations of unmodified and surface modified TiO₂—UCNs ranging from 10 μM to 1 mM for 6 h. The medium containing non-internalized nanoparticles were removed and the cells were washed thrice with PBS, and replaced with fresh culture medium. The cells were then irradiated using 980 nm NIR light at a power of 1.2 W for 5 min 20 sec delivering a total fluence of 675 J/cm². The cells were incubated for additional 24 h before percentage of cell viability relative to the control untreated cells was determined using CellTiter 96® AQueous One Solution Cell Proliferation Assay (Promega, Madison, Wis., USA) as per manufacturer's instructions.

Measurement of ROS production in vitro. The amount of ROS generated following in vitro PDT using TiO₂—UCNs or Mal-PEG-TiO₂—UCNs was measured using OxiSelect™ In vitro ROS/RNS Assay Kit (CellBiolabs, Inc. USA) following the manufacturer's instruction. Briefly, OSCC cells were seeded into 96-well cell culture plate at a cell density of 8×103 cells per well. Following overnight incubation at 37° C., the cells were then treated with TiO₂—UCNs or Mal-PEG-TiO₂—UCNs for 6 h, following which the medium was removed and replaced with 100 μL of the non-fluorescent 2′,7′-dichlorofluorescein (DCFH) for 1 h. The cells were then gently washed thrice with 1×PBS and fresh medium was added to each well followed by 980 nm NIR light irradiation using same light dose as mentioned above. The ROS generated in the cells was determined fluorometrically by measuring the amount of 2′, 7′-dichlorodihydrofluorescein (DCF) produced and comparing it with predetermined DCF standard curve.

Assessment of mode of cell-death. To study the mode of cell death following in vitro PDT, OSCC cells were lightly counterstained with 0.1% trypan blue in PBS for 5 min at different time intervals after treatment (30 min and 6 h). The cells were then gently washed once with 1×PBS and coverslipped with HBSS and immediately visualized using a bright field microscope fitted with a Nikon DS-Ri1 camera.

Statistical Analysis. In all figures, data points represent mean±standard deviation (SD). Statistical analyses were performed using the GraphPad Prism version 6.0 software (GraphPad Software, San Diego Calif. USA). Differences in means were compared with two-tailed unpaired Student's t-test or using two-way ANOVA followed by Bonferroni's post-hoc test. P values less than 0.05 (P<0.05) were considered significant.

### REFERENCES

1. Kenyon, A. J. Recent developments in rare-earth doped materials for optoelectronics. Progress in Quantum Electronics 26, 225-284 (2002).
2. Wang, F. & Liu, X. G. Upconversion multicolor fine-tuning: Visible to near-infrared emission from lanthanide-doped NaYF4 nanoparticles. Journal of the American Chemical Society 130, 5642-+(2008).
3. Heer, S., et al. Highly efficient multicolour upconversion emission in transparent colloids of lanthanide-doped NaYF4 nanocrystals. Advanced Materials 16, 2102-2105 (2004).
4. Li, Z. Q. & Zhang, Y. Monodisperse silica-coated polyvinylpyrrolidone/NaYF4 nanocrystals with multicolor upconversion fluorescence emission. Angewandte Chemie-International Edition 45, 7732-7735 (2006).
5. Idris, N. M., et al. Tracking transplanted cells in live animal using upconversion fluorescent nanoparticles. Biomaterials 30, 5104-5113 (2009).
6. Yu, M. X., et al. Laser Scanning Up-Conversion Luminescence Microscopy for Imaging Cells Labeled with Rare-Earth Nanophosphors. Anal. Chem. 81, 930-935 (2009).
7. Frangioni, J. V. In vivo near-infrared fluorescence imaging. Current Opinion in Chemical Biology 7, 626-634 (2003).
8. Palmer, R. J., et al. Cytotoxicity of the rare earth metals cerium, lanthanum, and neodymium in vitro: comparisons with cadmium in a pulmonary macrophage primary culture system. Environ Res 43, 142-156 (1987).
9. Saif, M. & Abdel-Mottaleb, M. S. A. Titanium dioxide nanomaterial doped with trivalent lanthanide ions of Tb, Eu and Sm: Preparation, characterization and potential applications. Inorganica Chimica Acta 360, 2863-2874 (2007).
10. Tang, Y. N., et al. NIR-Responsive Photocatalytic Activity and Mechanism of NaYF4:Yb,Tm@TiO2 Core-Shell Nanoparticles. Acs Catalysis 3, 405-412 (2013).
11. Xu, Q. C., et al. Anti-cAngpt14 Ab-Conjugated N—TiO2NaYF4:Yb,Tm Nanocomposite for Near Infrared-Triggered Drug Release and Enhanced Targeted Cancer Cell Ablation. Advanced Healthcare Materials 1, 470-474 (2012).
12. Zhang, D. S., et al. Synthesis and Upconversion Luminescence of YF3:Yb3+,Tm3+ and TiO₂—Coated YF3:Yb3+,Tm3+ Microcrystals. Journal of Nanoscience and Nanotechnology 10, 2032-2035 (2010).
13. Idris, N. M., et al. In vivo photodynamic therapy using upconversion nanoparticles as remote-controlled nanotransducers. Nature Medicine 18, 1580-U1190 (2012).
14. Guo, H. C., et al. Singlet oxygen-induced apoptosis of cancer cells using upconversion fluorescent nanoparticles as a carrier of photosensitizer. Nanomedicine-Nanotechnology Biology and Medicine 6, 486-495 (2010).
15. Qian, H., et al. Mesoporous-Silica-Coated Up-Conversion Fluorescent Nanoparticles for Photodynamic Therapy. Small 5, 2285-2290 (2009).
16. Suttiponparnit, K.; Jiang, J.; Sahu, M.; Suvachittanont, S.; Charinpanitkul, T.; Biswas, P., Role of Surface Area, Primary Particle Size, and Crystal Phase on Titanium Dioxide Nanoparticle Dispersion Properties. Nanoscale Res. Lett. 2011, 6, 27.
17. Qi, L.; Xu, Z.; Jiang, X.; Hu, C.; Zou, X., Preparation and Antibacterial Activity of Chitosan Nanoparticles. Carbohydr. Res. 2004, 339, 2693-2700.
18. Lundqvist, M.; Stigler, J.; Elia, G.; Lynch, I.; Cedervall, T.; Dawson, K. A., Nanoparticle Size and Surface Properties Determine the Protein Corona with Possible Implications for Biological Impacts. Proc. Natl. Acad. Sci. 2008, 105, 14265-14270.
19. Klinger, A.; Steinberg, D.; Kohavi, D.; Sela, M. N., Mechanism of Adsorption of Human Albumin to Titanium In vitro. J. Biomed. Mater. Res. 1997, 36, 387-392.
20. Aggarwal, P.; Hall, J. B.; McLeland, C. B.; Dobrovolskaia, M. A.; McNeil, S. E., Nanoparticle Interaction with Plasma Proteins as It Relates to Particle Biodistribution, Biocompatibility and Therapeutic Efficacy. Adv. Drug Deliv. Rev. 2009, 61, 428-437.
21. Wolfram, J.; Yang, Y.; Shen, J.; Moten, A.; Chen, C.; Shen, H.; Ferrari, M.; Zhao, Y., The Nano-Plasma Interface: Implications of the Protein Corona. Colloids Surf., B 2014, DOI: 10.1016/j.colsurfb.2014.02.035.
22. Vonarbourg, A.; Passirani, C.; Saulnier, P.; Benoit, J. P., Parameters Influencing the Stealthiness of Colloidal Drug Delivery Systems. Biomaterials 2006, 27, 4356-4373.
23. Owens, D. E., 3rd; Peppas, N. A., Opsonization, Biodistribution, and Pharmacokinetics of Polymeric Nanoparticles. Int. J. Pharm. 2006, 307, 93-102.
24. Jokerst, J. V.; Lobovkina, T.; Zare, R. N.; Gambhir, S. S., Nanoparticle Pegylation for Imaging and Therapy. Nanomedicine 2011, 6, 715-728.
25. Sakhtianchi, R.; Minchin, R. F.; Lee, K.-B.; Alkilany, A. M.; Serpooshan, V.; Mahmoudi, M., Exocytosis of Nanoparticles from Cells: Role in Cellular Retention and Toxicity. Adv. Colloid Interface Sci. 2013, 201-202, 18-29.
26. Bae, Y. M.; Park, Y. I.; Nam, S. H.; Kim, J. H.; Lee, K.; Kim, H. M.; Yoo, B.; Choi, J. S.; Lee, K. T.; Hyeon, T., et al., Endocytosis, Intracellular Transport, and Exocytosis of Lanthanide-Doped Upconverting Nanoparticles in Single Living Cells. Biomaterials 2012, 33, 9080-9086.
27. Li, T.; Takeoka, S., A Novel Application of Maleimide for Advanced Drug Delivery: In vitro and In vivo Evaluation of Maleimide-Modified pH-Sensitive Liposomes. Int. J. Nanomedicine 2013, 8, 3855-3866.
28. Li, T.; Takeoka, S., Enhanced Cellular Uptake of Maleimide-Modified Liposomes via Thiol-Mediated Transport. Int. J. Nanomedicine 2014, 9, 2849-2861.
29. Liu, K.; Liu, X.; Zeng, Q.; Zhang, Y.; Tu, L.; Liu, T.; Kong, X.; Wang, Y.; Cao, F.; Lambrechts, S. A., et al., Covalently Assembled NIR Nanoplatform for Simultaneous Fluorescence Imaging and Photodynamic Therapy of Cancer Cells. ACS Nano 2012, 6, 4054-4062.
30. Ripamonti, F.; Albano, L.; Rossini, A.; Borrelli, S.; Fabris, S.; Mantovani, R.; Neri, A.; Balsari, A.; Magnifico, A.; Tagliabue, E., EGFR through Stat3 Modulates Δn63α Expression to Sustain Tumor-Initiating Cell Proliferation in Squamous Cell Carcinomas. J. Cell. Physiol. 2013, 228, 871-878.
31. Buytaert, E.; Dewaele, M.; Agostinis, P., Molecular Effectors of Multiple Cell Death Pathways Initiated by Photodynamic Therapy. Biochim. Biophys. Acta, Rev. Cancer 2007, 1776, 86-107.
32. Mroz, P.; Yaroslaysky, A.; Kharkwal, G. B.; Hamblin, M. R., Cell Death Pathways in Photodynamic Therapy of Cancer. Cancers 2011, 3, 2516-2539.
33. Henderson, B. W.; Dougherty, T. J., How Does Photodynamic Therapy Work? Photochem. Photobiol. 1992, 55, 145-157.
34. Dolmans, D. E. J. G. J.; Fukumura, D.; Jain, R. K., Photodynamic Therapy for Cancer. Nat. Rev. Cancer 2003, 3, 380-387.
35. Oleinick, N. L.; Morris, R. L.; Belichenko, I., The Role of Apoptosis in Response to Photodynamic Therapy: What, Where, Why, and How. Photochem. Photobiol. Sci. 2002, 1, 1-21.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A nanocomposite for photodynamic therapy, comprising:
an upconversion nanoparticle, wherein the nanoparticle, upon excitation by near infrared light, emits light of a wavelength from about 330 nm to about 675 nm; and
a continuous and uniform outer coating on the outer surface of the nanoparticle, the coating comprising a semiconductor material, wherein the light emitted from the nanoparticle is of a wavelength sufficient to excite one or more electrons from a valence band of the semiconductor material to the conduction band of the semiconductor material, and the semiconductor material, after excitation, is of an energy sufficient to generate at least one reactive oxygen species.

2. The nanocomposite of claim 1, wherein the nanoparticle comprises NaYF₄ nanocrystals doped with from about 10 mole % to about 30 mole % Yb³⁺ and from about 0.3 mole % to about 2 mole % Tm³⁺.

3. The nanocomposite of claim 2, wherein the Y:Yb:Tm molar ratio is 79.5:20:0.5.

4. The nanocomposite of claim 1, wherein the semiconductor material is TiO₂.

5. The nanocomposite of claim 1, further comprising an intermediate coating layer, wherein the intermediate coating layer is positioned between the upconversion nanoparticle and the outer coating comprising the semiconductor material.

6. The nanocomposite of claim 1, wherein the nanocomposite is modified with a targeting agent.

7. The nanocomposite of claim 6, wherein the targeting agent is linked to the semiconductor surface through a linking group.

8. The nanocomposite of claim 7, wherein the linking group is poly(ethylene)glycol.

9. The nanocomposite of claim 8, wherein the targeting agent is an affibody, an antibody, an aptameror a peptide.

10. A composition for photodynamic therapy, comprising:
a targeting agent bound to a scaffold comprising a nanocomposite, wherein the nanocomposite comprises:
an upconversion nanoparticle, wherein the nanoparticle, upon excitation by near infrared light, emits light of a wavelength from about 330 nm to about 675 nm; and
a continuous coating on the outer surface of the nanoparticle, the coating comprising a semiconductor material, wherein the light emitted from the nanoparticle is of a wavelength sufficient to excite one or more electrons from a valence band of the semiconductor material to the conduction band of the semiconductor material, and the semiconductor material, after excitation, is of an energy sufficient to generate at least one reactive oxygen species.

11. The composition of claim 10, wherein the targeting agent is linked to the semiconductor surface, optionally through a linking group.

12. A method of generating reactive oxygen species, comprising:
irradiating with near infrared light a sample comprising the nanocomposite of claim 1 and one or more oxygen sources selected from water or oxygen for a period of time sufficient to excite one or more electrons from a valence band of the semiconductor material to the conduction band of the semiconductor material, wherein the one or more oxygen sources undergoes a redox reaction to form a reactive oxygen species.

13. A nanocomposite composition, comprising:
a plurality of nanocomposites of claim 1, wherein the nanocomposites are uniformly distributed throughout the composition, and further wherein the nanocomposites are uniform in shape and size.

14. The nanocomposite of claim 7, wherein the linking group is a dispersion stabilizer.

15. The nanocomposite of claim 14, wherein the dispersion stabilizer is PEG.

16. The nanocomposite of claim 15, wherein the molecular weight of the dispersion stabilizer is 2000 Da or greater.

17. A method of administering photodynamic therapy to treat a biological target in a subject, the method comprising:
administering a therapeutically effective amount of the nanocomposite of claim 1 to the subject;
exposing the nanocomposite to near infrared light sufficient to cause the nanocomposite particle to emit light of a wavelength of about 330 nm to about 675 nm such that the generated at least one reactive oxygen species treats the biological target in the subject.

18. The method of claim 17, wherein the biological target is a cell surface receptor that is overexpressed in a cancerous cell.
